(19) United States
(12) Patent Application Publication
(10) Pub. No.: US 2024/0257987 A1
(43) Pub. Date: Aug. 1, 2024

(54) CLINICIAN VIDEO EFFICIENCY SYSTEM

(71) Applicant: Video Portal Concepts LLC, Santa Fe, NM (US)

(72) Inventor: Shawn Bidot, Phoenix, AZ (US)

(21) Appl. No.: 18/160,387

(22) Filed: Jan. 27, 2023

(51) Int. Cl. G16H 80/00; G16H 40/20

(52) U.S. Cl. CPC G16H 80/00; G16H 40/20

(57) ABSTRACT

An educational Video Patient Portal (VPP) that is part of a Clinician Video Efficiency System (CVES) is provided, as well as new processes for building, utilizing, optimizing, customizing, or improving a VPP and a CVES. Doctors, clinicians, and/or healthcare providers can create their own personal video content from their cell phones, computers, video recorders, or other recording devices on different health-care related topics that they feel are important to their patients, enhance their practice, and/or are discussed frequently at their office with their patients. These videos can be uploaded into a private secure and proprietary VPP. The library of personally created video content created by the doctor, clinician, and/or healthcare provider can be watched by their patients for free at any time, including at home, in an exam room and/or before seeing the doctor, clinician or healthcare provider.

### BACKGROUND

Healthcare providers can spend a significant amount of time each day communicating educational information to patients. This time then takes away from time available for each patient interaction. While large amounts of healthcare information can be found from publicly available sources, public domain health information is not controlled by the healthcare provider, and therefore can be out of date, inconsistent, unreliable, incomplete, incorrect, or even harmful. Additionally, such public domain health information can actually compete with and not enhance the relationship and trust between the healthcare provider and patient.

### BRIEF SUMMARY

Embodiments of the subject invention provide an educational Video Patient Portal (VPP) that is part of a Clinician Video Efficiency System (CVES). Embodiments provide new processes for building, utilizing, optimizing, customizing, or improving a VPP and a CVES.

In certain embodiments doctors, clinicians, and/or healthcare providers create their own personal video content from their cell phones, computers, video recorders, or other recording devices on different health-care related topics (e.g., topics that they feel are important to their patients, their practice, and/or normally discussed regularly, frequently, weekly, or daily at their office with their patients.) These videos are then uploaded into a private secure and proprietary VPP. The library of personally created video content created by the doctor, clinician, and/or healthcare provider can be watched by their patients for free at any time, including at home, in an exam room and/or before seeing the doctor, clinician or healthcare provider.

### BRIEF DESCRIPTION OF DRAWINGS

FIG. 1A is a schematic flow chart of a process utilizing a VPP and the CVES according to an embodiment of the subject invention.

FIG. 1B is a schematic diagram of a system for providing a VPP and the CVES to support an individual patient interaction according to an embodiment of the subject invention.

FIG. 1C is a schematic flow chart of a process utilizing a VPP supported by the CVES to support a series of patient interactions with a nurse or other healthcare provider in a telephone communication setting according to an embodiment of the subject invention.

FIG. 1D is a schematic flow chart of a process utilizing a VPP supported by the CVES to support a series of patient interactions with a doctor or other healthcare provider in a multiple exam room setting according to an embodiment of the subject invention.

FIG. 2 is a screenshot showing a portal login landing page according to an embodiment of the subject invention.

FIG. 3 is a screenshot showing a patient sign in page according to an embodiment of the subject invention.

FIG. 4 is a screenshot showing a patient disclaimer page according to an embodiment of the subject invention.

FIG. 5 is a screenshot showing a patient portal home page according to an embodiment of the subject invention.

FIG. 6 is a screenshot showing a doctor profile page according to an embodiment of the subject invention.

FIG. 7 is a screenshot showing a patient video selection page according to an embodiment of the subject invention.

FIG. 8 is a screenshot showing a patient video viewing page according to an embodiment of the subject invention.

FIG. 9 is a screenshot showing a patient forms page according to an embodiment of the subject invention.

FIG. 10 is a screenshot showing a patient links page according to an embodiment of the subject invention.

FIG. 11 is a screenshot showing a doctor sign in page according to an embodiment of the subject invention.

FIG. 12 is a screenshot showing a doctor sign in two factor authentication page according to an embodiment of the subject invention.

FIG. 13 is a screenshot showing a doctor dashboard page according to an embodiment of the subject invention.

FIG. 14 is a screenshot showing a doctor account settings page according to an embodiment of the subject invention.

FIG. 15 is a screenshot showing a doctor profile page according to an embodiment of the subject invention.

FIG. 16 is a screenshot showing a doctor video selection page according to an embodiment of the subject invention.

FIG. 17 is a screenshot showing a doctor forms page according to an embodiment of the subject invention.

FIG. 18 is a screenshot showing a doctor links page according to an embodiment of the subject invention.

FIG. 19 is a screenshot showing a payment process sign up page according to an embodiment of the subject invention.

FIG. 20 is a screenshot showing a payment process sign up phone number verification page according to an embodiment of the subject invention.

FIG. 21 is a screenshot showing a payment process sign up two factor authentication page according to an embodiment of the subject invention.

FIG. 22 is a screenshot showing a payment process sign up promotional code page according to an embodiment of the subject invention.

FIG. 23 is a screenshot showing a payment process sign up plan selection page according to an embodiment of the subject invention.

FIG. 24 is a screenshot showing a payment process sign up payment details page according to an embodiment of the subject invention.

FIG. 25 is a screenshot showing a payment process payment information page according to an embodiment of the subject invention.

FIG. 26 is a screenshot showing a payment process secure checkout page according to an embodiment of the subject invention.

FIG. 27 is a screenshot showing a payment process sign up completed page according to an embodiment of the subject invention.

### DETAILED DESCRIPTION

Embodiments of the subject invention provide an educational VPP that is part of a CVES. Embodiments provide a CVES configured and adapted to provide a system in one or more health care provider (HCP) offices that increases time efficiency and overall practice efficiency by utilizing a library of personally created video content created by a doctor, clinician, and/or healthcare provider. By watching the doctor, clinician, and/or healthcare provider's videos, the patients become better informed on the topic of interest, and gain an enhanced relationship with their specific healthcare provider. According to certain embodiments, when patients watch a video of their own doctor, clinician, and/or healthcare provider they will be better educated on the topic of interest before they have the office visit appointment. This gives the patient time to formulate any questions prior to the office visit, makes the patient interaction more focused, and minimizes patient confusion compared to other sources of online content. Numerous purposes, usages, benefits, and advantages of the VPP and CVES according to embodiments of the subject invention are presented below.

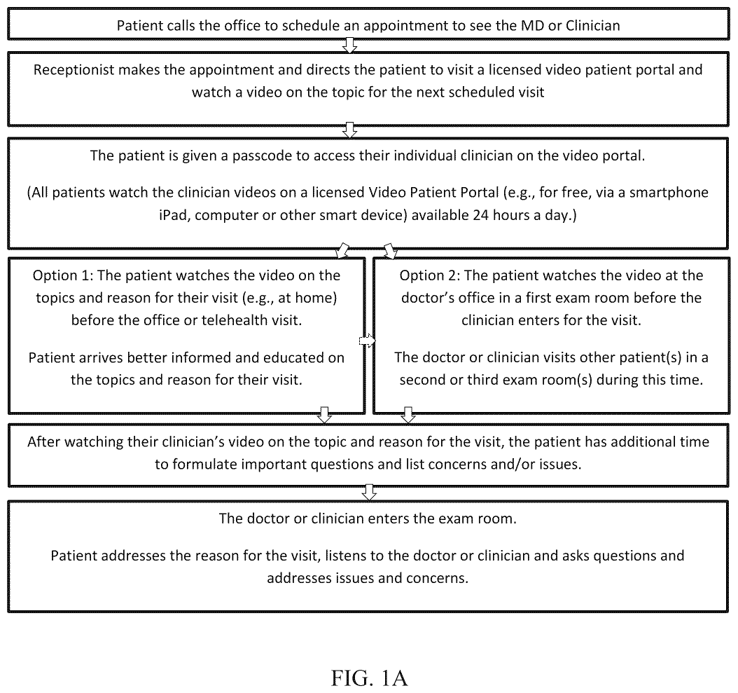

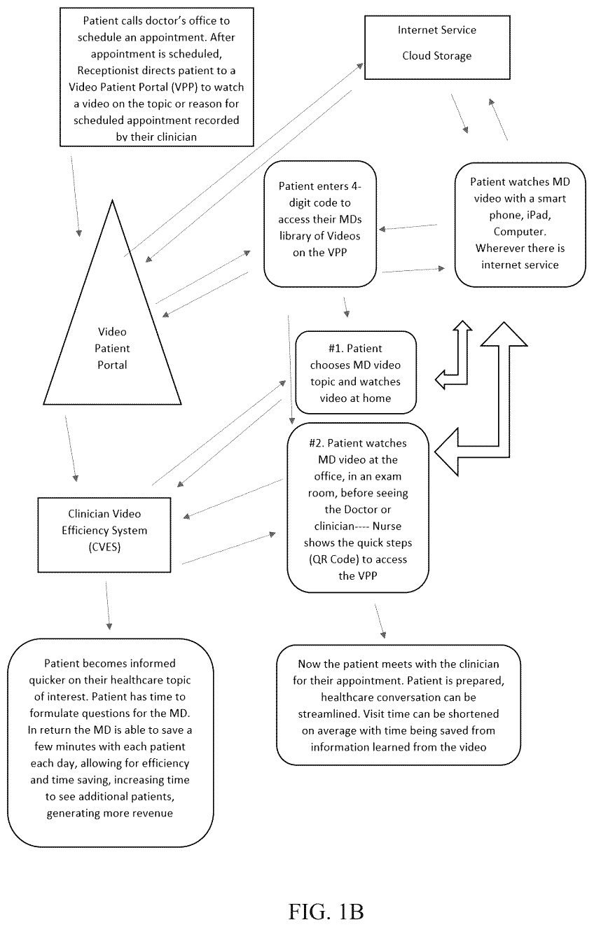

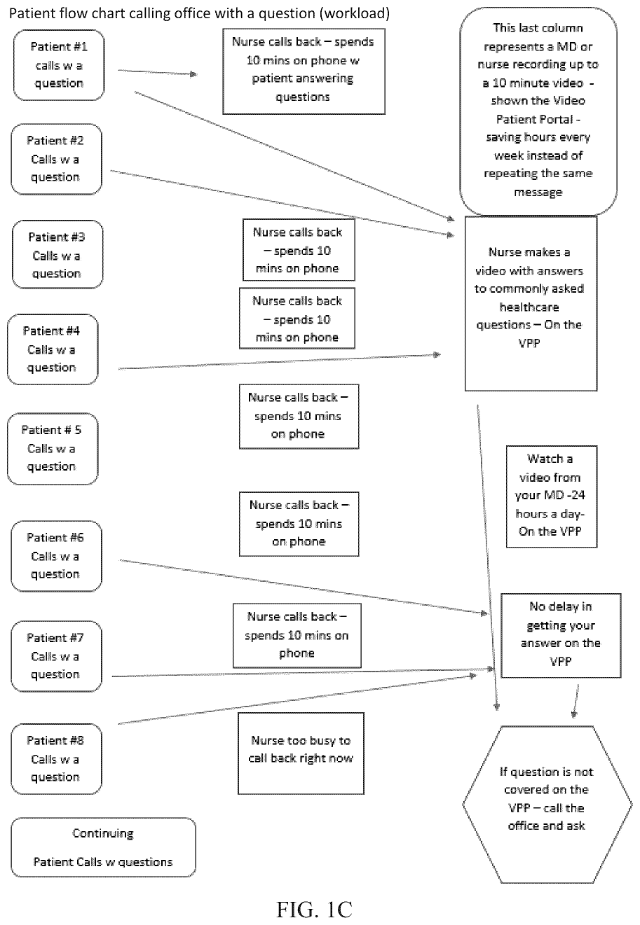

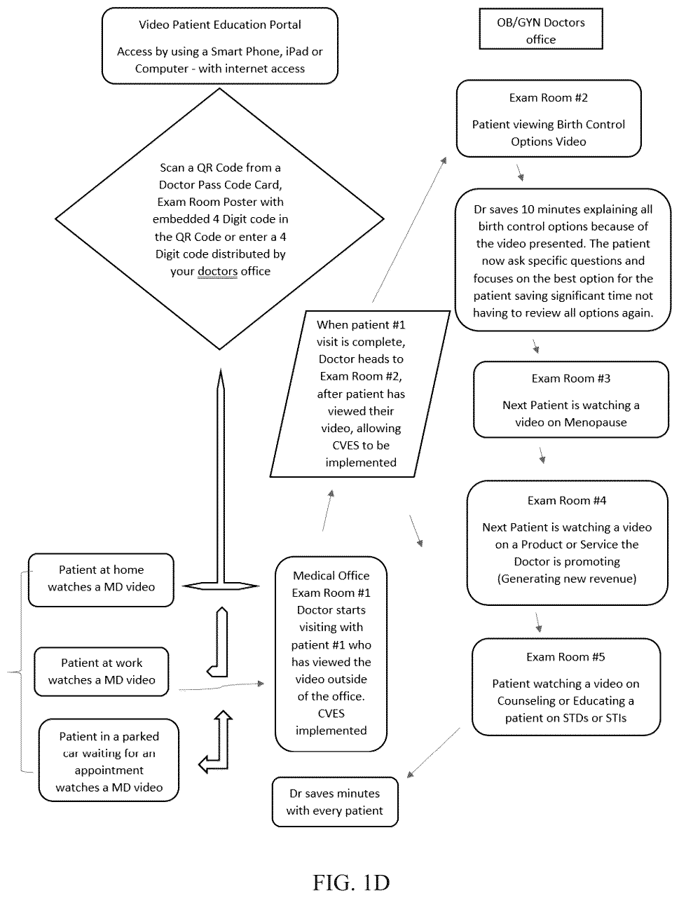

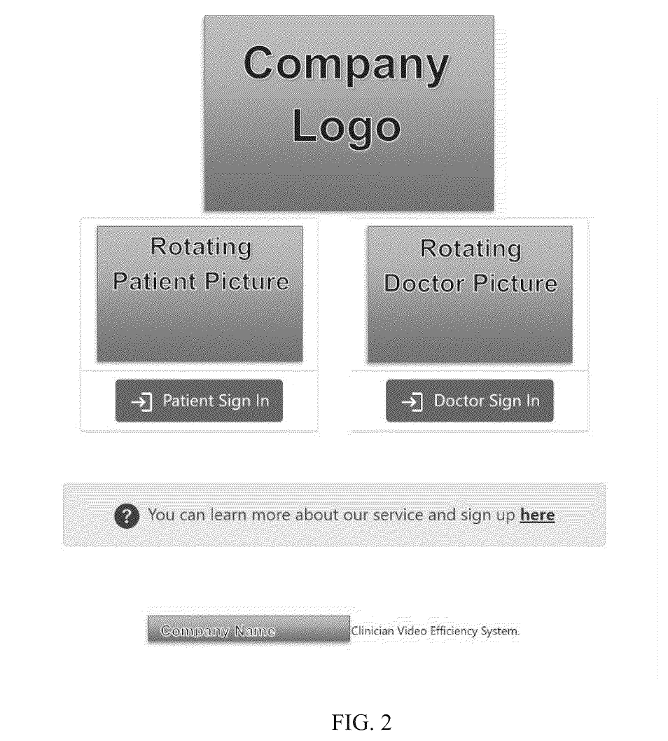

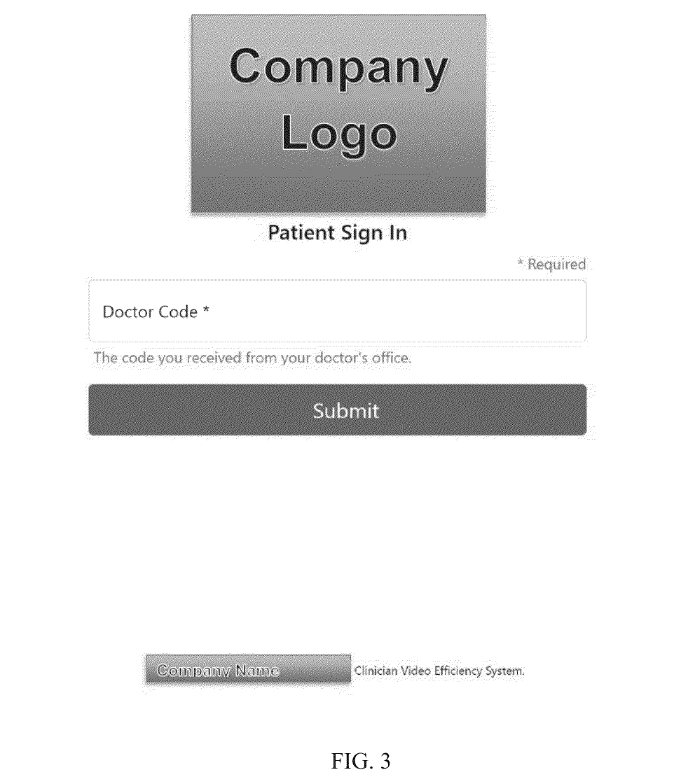

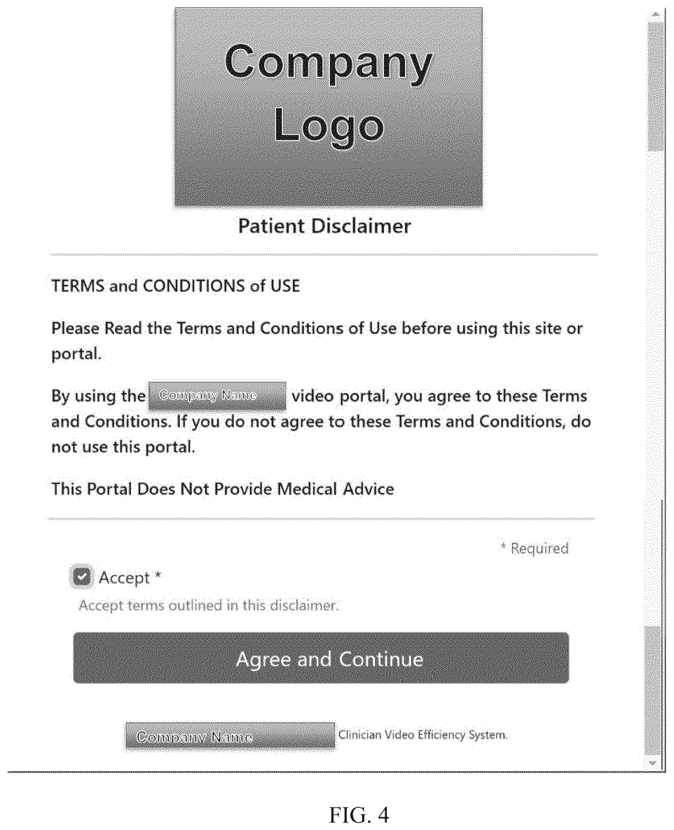

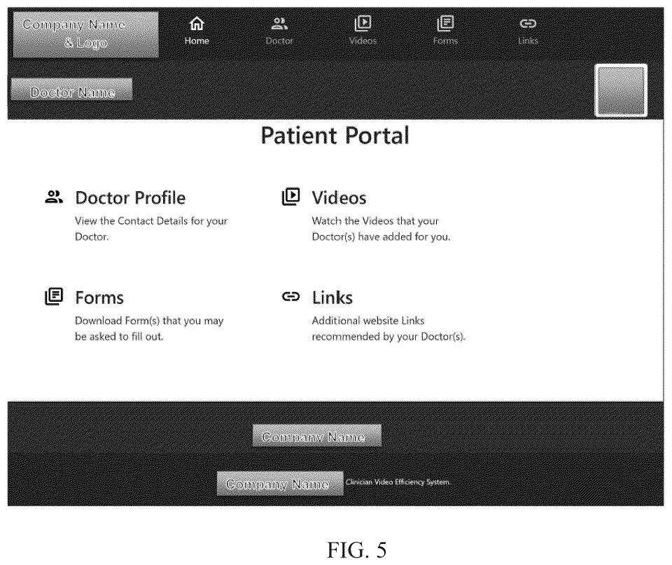

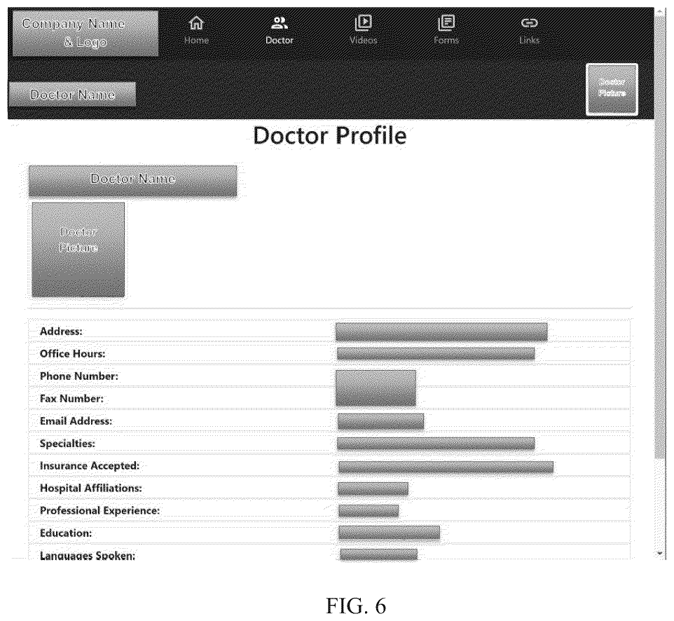

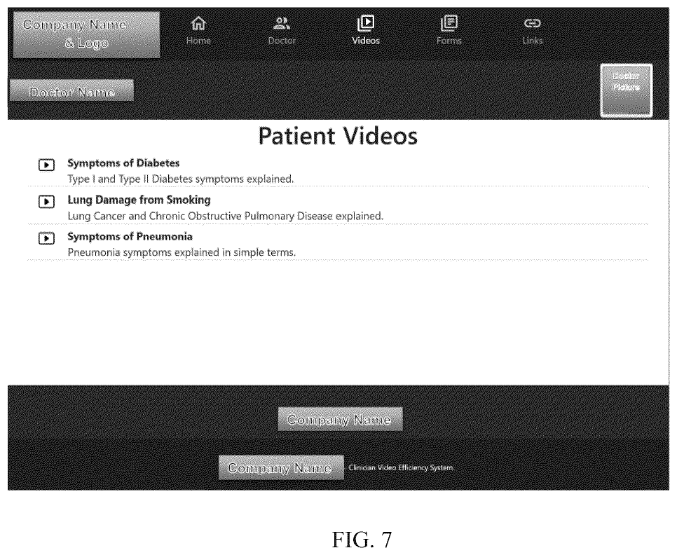

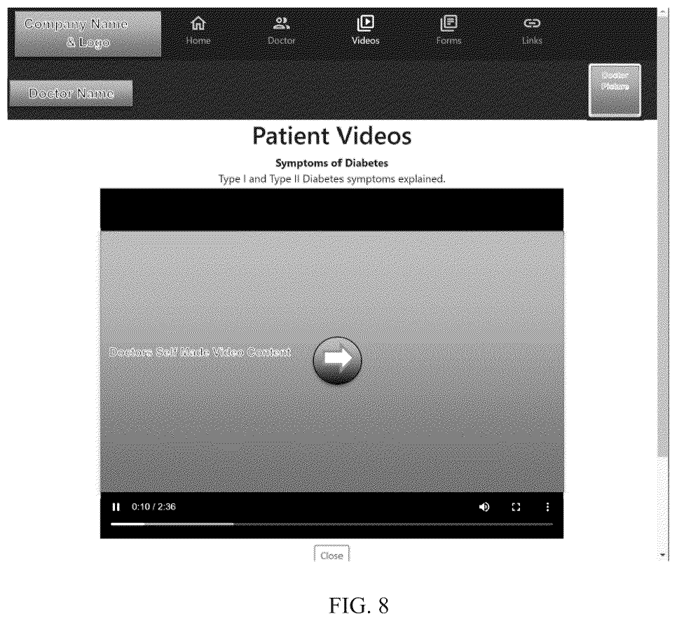

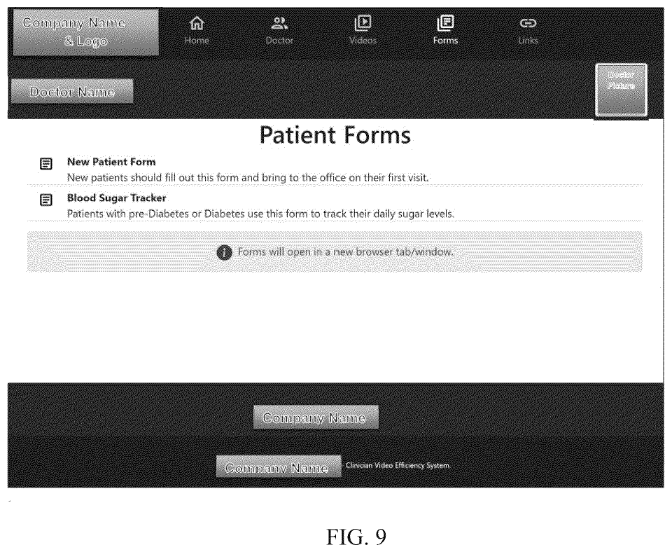

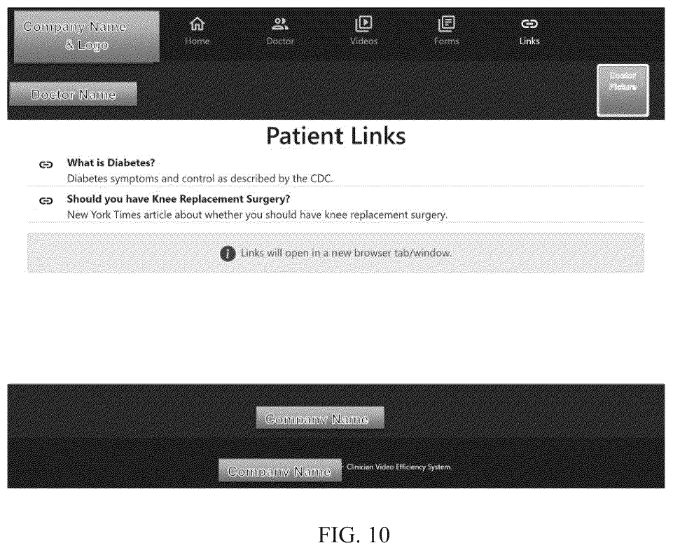

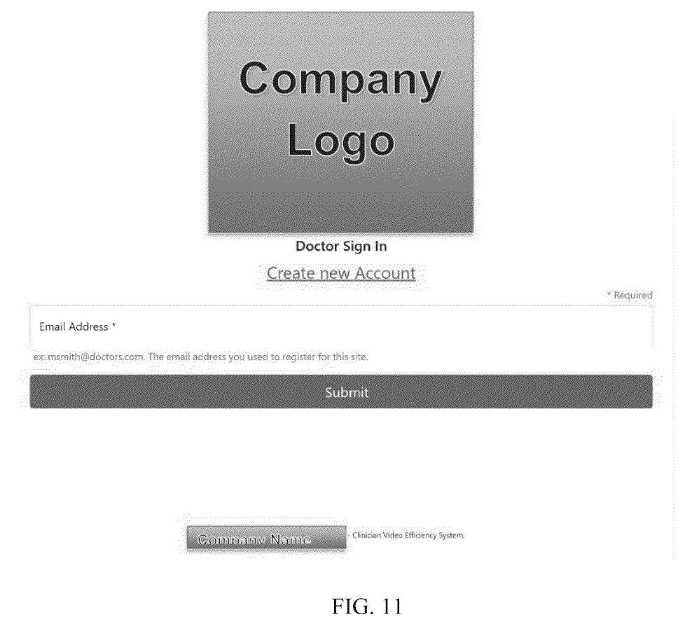

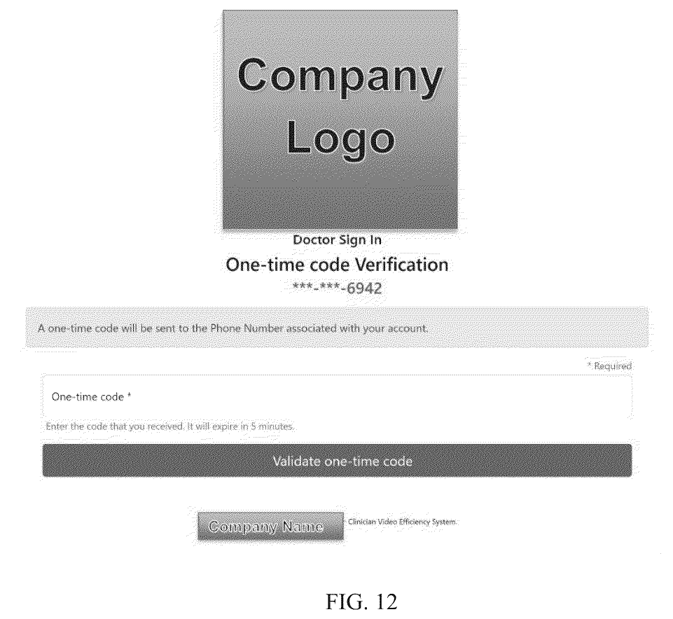

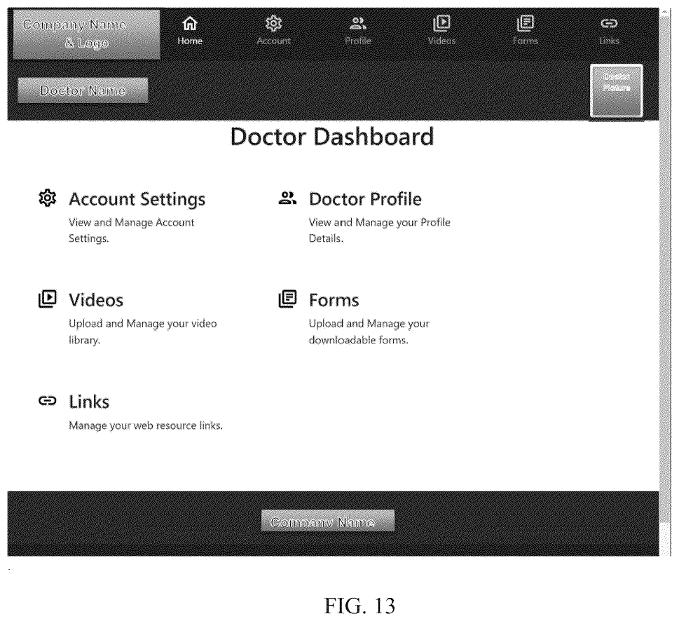

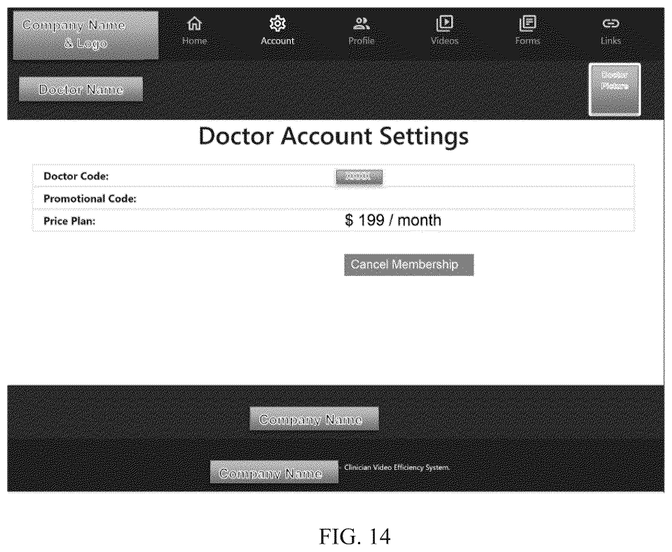

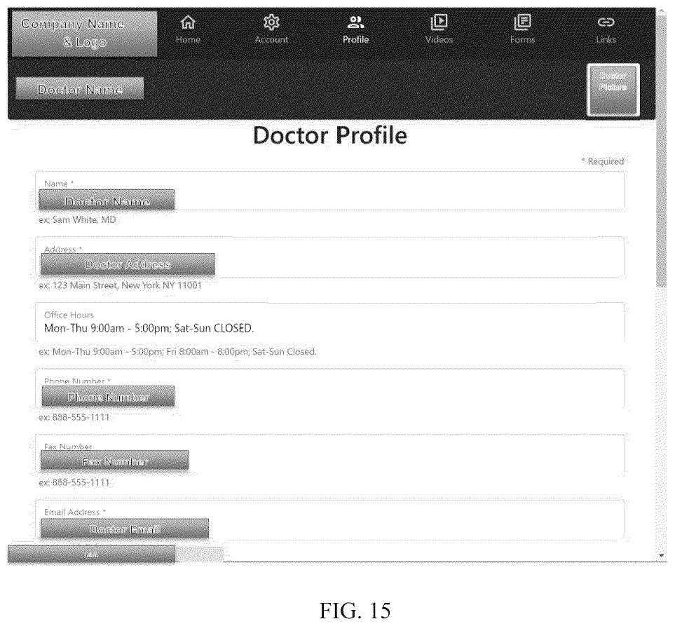

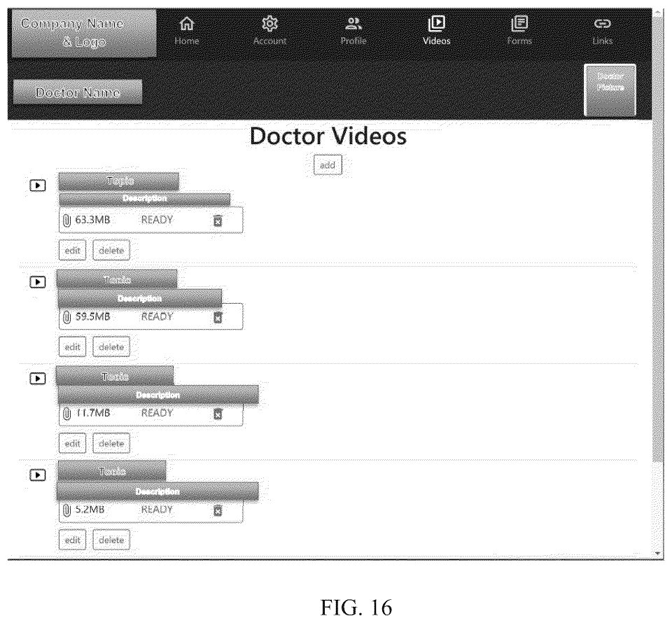

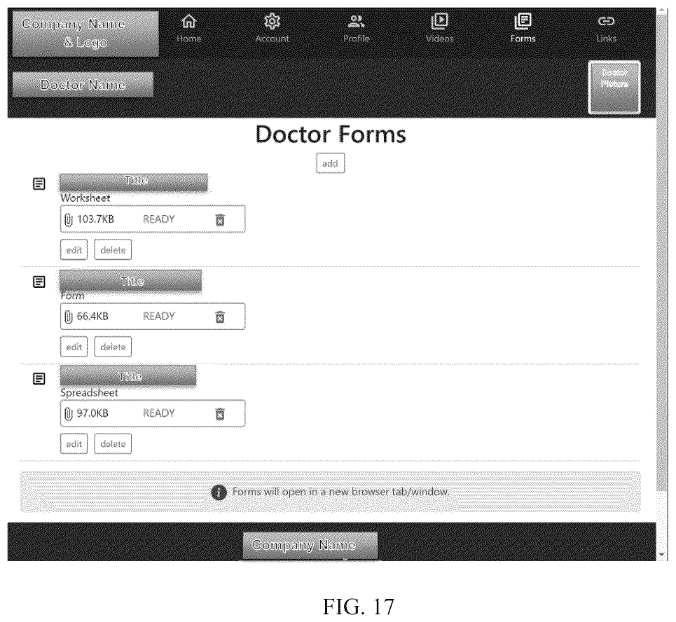

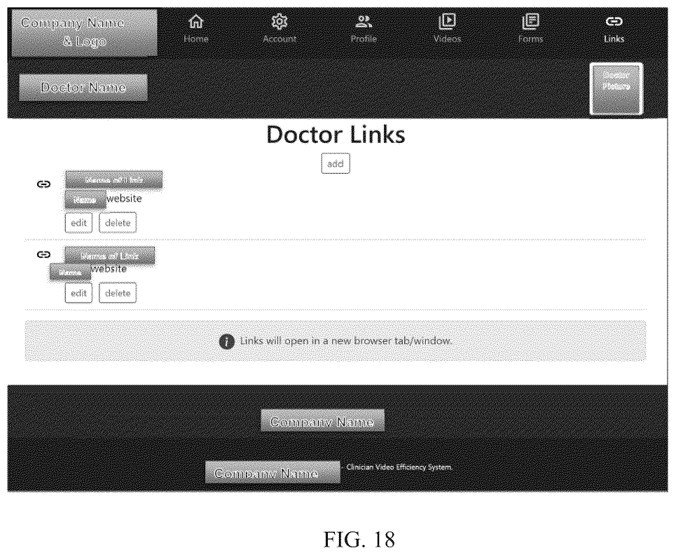

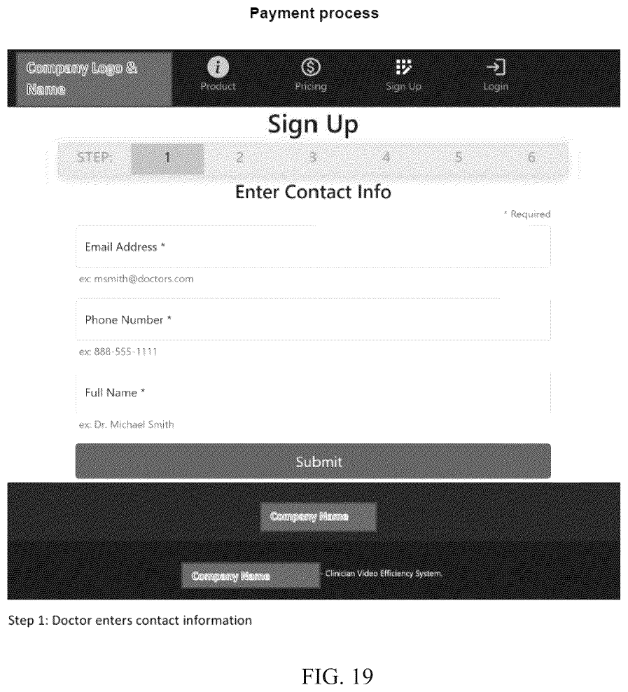

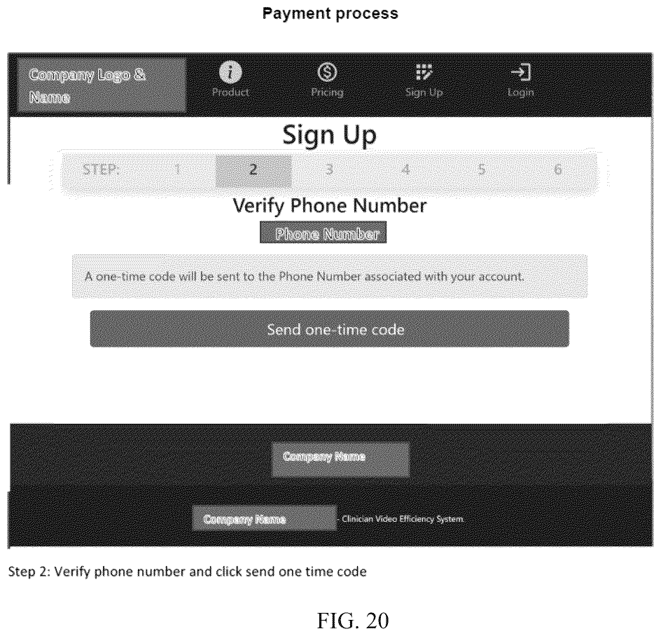

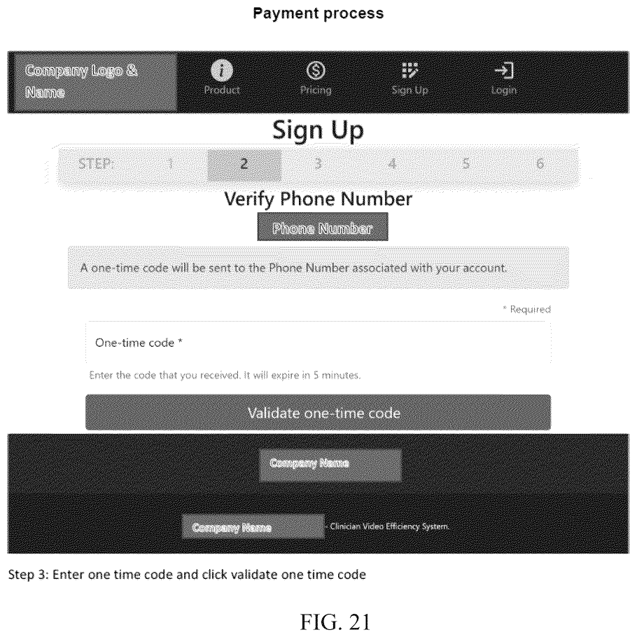

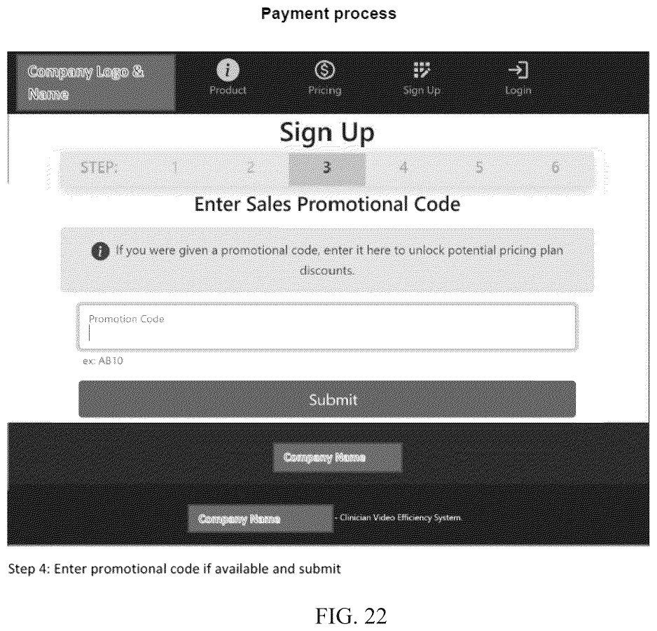

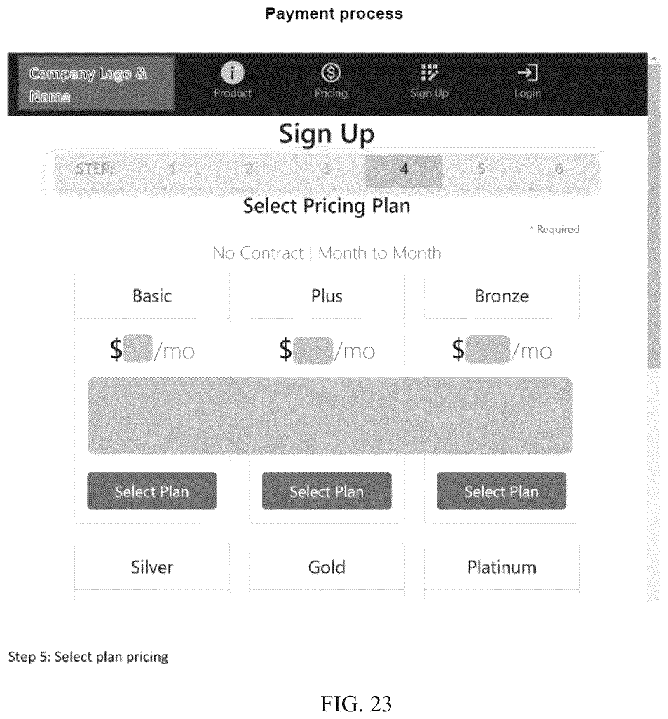

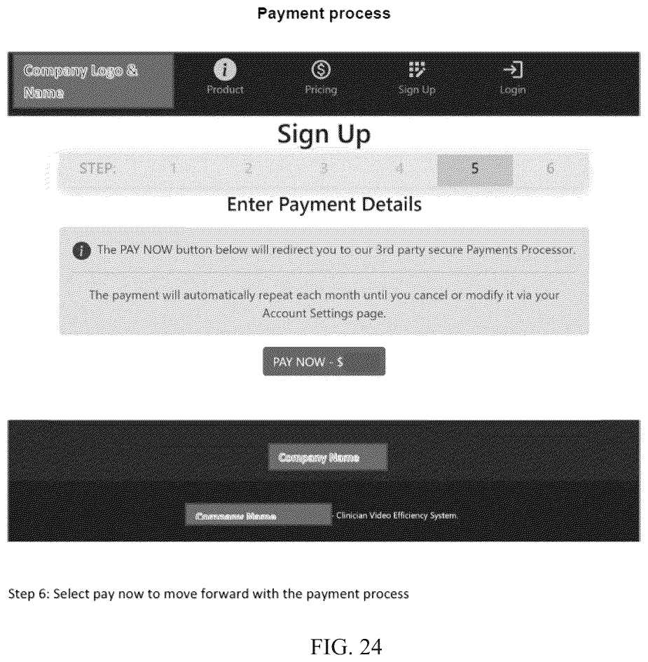

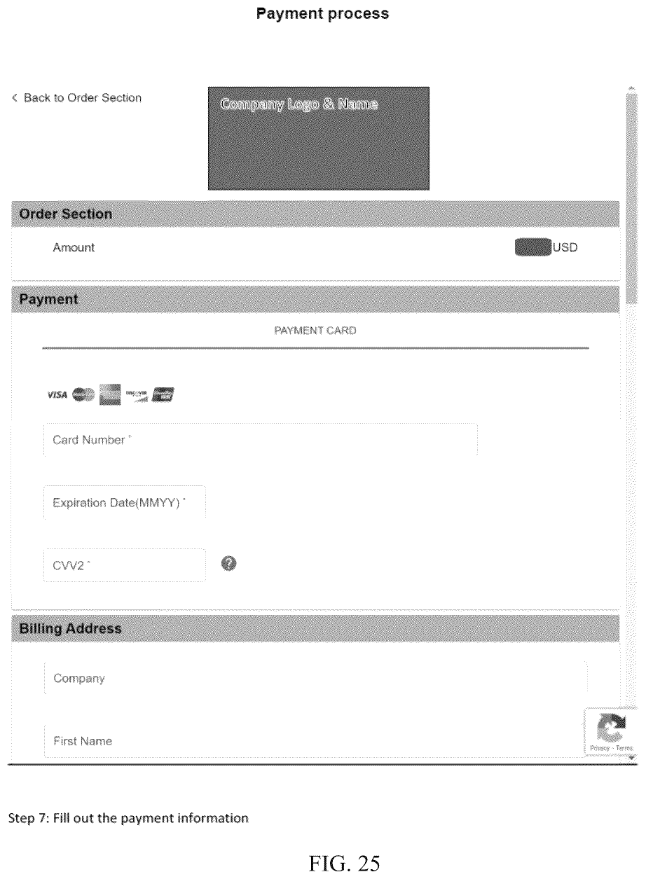

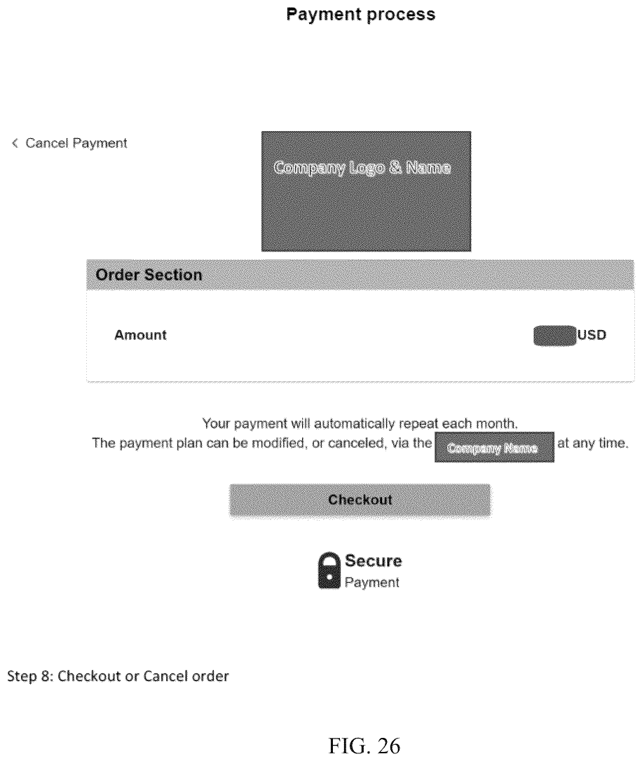

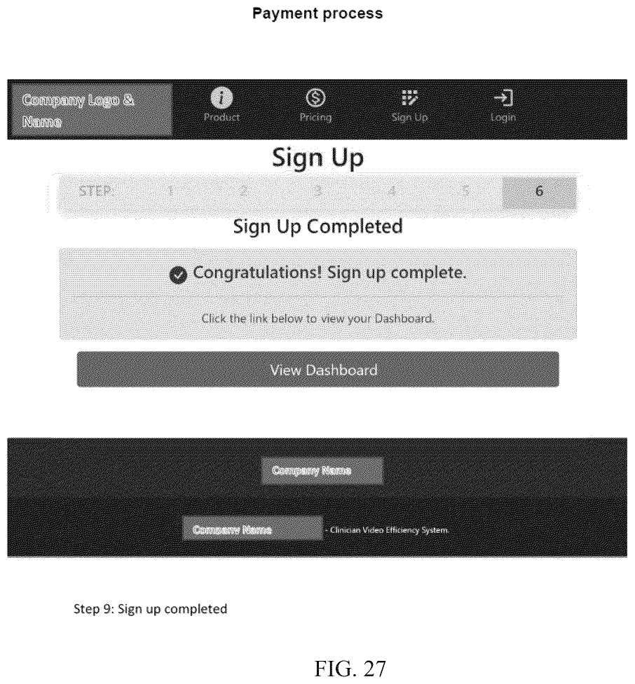

FIG. 1A shows a schematic flow chart of a process utilizing a VPP and the CVES, according to an embodiment of the subject invention; FIG. 1B shows a schematic diagram of a system for providing a VPP and the CVES to support an individual patient interaction, according to an embodiment of the subject invention; FIG. 1C shows a schematic flow chart of a process utilizing a VPP supported by the CVES to support a series of patient interactions with a nurse or other healthcare provider in a telephone communication setting, according to an embodiment of the subject invention; and FIG. 1D shows a schematic flow chart of a process utilizing a VPP supported by the CVES to support a series of patient interactions with a doctor or other healthcare provider in a multiple exam room setting, according to an embodiment of the subject invention. FIGS. 2-27 show various screenshots of the VPP and/or CVES, according to embodiments of the subject invention.

Embodiments of a CVES according to the subject invention can be used in the healthcare setting to allow doctors, clinicians, and nursing staff to save time with their patients at the office or when patients are calling the office on the phone by utilizing an educational VPP. Patients can watch healthcare videos recorded by their MD and get information before the doctor's office visit. Embodiments allow medical doctors (MDs) and clinicians or other healthcare providers to inform patients more quickly, effectively, and efficiently on topics of interest and move the healthcare conversation forward at the office with the time saved. Additional revenue can be generated with that time saved by seeing additional patients each day.

In certain embodiments (e.g., by way of example and not limitation, as shown in FIG. 1B) the VPP is part of the CVES and can be defined as a tool of CVES. The VPP is very important to the success of CVES in certain embodiments because as a number of custom-made videos are added into the MD's Library of topics, the VPP increases the ability to have an impact on each patient-MD interaction, or clinician office visit, or phone call-back. When a healthcare video has been recorded on a topic or subject that affects a patient, there is an opportunity to inform and educate the patient before the office visit appointment. In certain embodiments the video can be less than 10 minutes. The patient can gain more information by the dual process of watching a video of their healthcare provider and then a conversation with their healthcare provider. The patient can re-visit the video to clarify a point their healthcare provider made. Embodiments help patients on multiple fronts, and advantageously allow MDs to structure appointments to be more fact filled, patient engaging, and time efficient. Embodiments of CVES including VPP can be a solution for an overburdened healthcare system. Embodiments allow MDs and clinicians to have the effectiveness of providing the benefits of patient interaction in more than one place at a time. In certain embodiments (e.g., by way of example and not limitation, as shown in FIG. 1B) the system can have its greatest positive outcomes through advantageous integration of a multiplicity of components, connections, and/or processes. In certain embodiments the CVES and VPP can be either a physically connected system, a logically connected system, or both. Embodiments of the subject invention provide a CVES, that when followed, from a numerical process will allow the HCP to save time in the Medical Doctors Office (MDO) or other healthcare setting. The utilization of the VPP as a tool works in support of CVES to distribute the MD's or HCP's video messages providing healthcare answers to common inquiries. These healthcare videos can have a greater impact when the video topics have a greater accuracy in interest and inquiries from the patient population. When treating a patient, the more video topics that can educate a patient on their concern or interest will increase the level of success with CVES. When an HCP can employ more video topics to a patient population then the HCP will have a higher level of success in saving time due to more expansive coverage with respect to healthcare topic needs of patients.

When an HCP or MD applies an embodiment comprising CVES in the exam room after a patient has watched the video of interest, they can have greater control of the structure of the patient visit. This can include reviewing the information on the video; however, it also can include reducing or eliminating some of the redundancy or basic information shared in the beginning. The patient can still ask questions but can be further along in the conversation with a comfort level in knowing that they can review some information on their mobile device later if, for example, they are concerned that they may have forgotten some of the details. The patient gets more of the information they need, and the MD can save a few minutes in the visit.

According to certain embodiments nurses and receptionist being able to deploy the healthcare videos can increase efficiency at the office when patients call with issues or concerns requiring answers. Having a video on the most inquired topics allows for greater impact on outcomes with a larger patient population. If, for example, 20 patients inquire about diabetes information after office hours one week but are directed to the VPP where they gain insight into their treatment, diet needs, and exercise suggestions, then this viewing can lead to positive outcomes. The challenged patient can watch a video multiple times of their MD or clinician which can carry more credibility than some unknown person or MD on the internet. The viewing in the privacy of their home in a safe area can be the opportunity for a MD or Clinician to have an impact in a patient's healthcare needs.

When patients are in the exam rooms posters can be provided on the wall showing the VPP, and can have a QR Code that represents the individual MD or Clinician. When the patient or nurse scans the code the MD or Clinician videos can appear. This quick, easy access can lead to more success due to ease of use and user friendliness.

In some embodiments Doctor Passcode cards can be distributed as a patient leaves for future use and access can then be provided, for free, 24 hours a day.

In embodiments where some or all of the above features and actions are provided, CVES and the VPP can provide a system that can be employed in private practices, Clinics, and healthcare settings.

In certain embodiments the VPP runs on or is configured and adapted to run on Amazon Cloud infrastructure or alternatively, on any available public cloud service or infrastructure.

In certain embodiments there are no additional networks or servers required to run outside of the cloud. The patient or doctors can interreact with the VPP hosted by Amazon Cloud. The VPP is accessible from any mobile device, computer, or device with internet access.

Embodiments of CVES provide a system that uses VPP to educate and instruct patients. Embodiments can be available around the clock but can also save MD, Clinicians, nurses, and office staff time in the office. The increase in efficiency can lead to significant time saving with patients and now additional patients can be seen in the office generating new revenue.

According to certain embodiments, multiple people can use the VPP at the same time, subject generally to practical or functional limits (e.g., server hardware, network bandwidth, storage, or the like.) If a patient has access to a smart phone, iPad, or computer with internet access they can have access to the VPP, from a multitude of locations. In certain embodiments CVES includes a system or program implemented at the healthcare office or clinic that allows for efficiency in a healthcare setting when seeing a high-volume of patients. When CVES uses VPP it allows patients to get healthcare information quicker on their topic of interest before coming into the office or before seeing the doctor in the exam room. The VPP can be one part of a multi part process when going to a medical office. CVES allows the patient to front load healthcare information they would otherwise receive at the office visit. Utilizing embodiments of CVES and a VPP the patient receives information on the topic (e.g., a short video—up to 10 minutes long, from their clinician) for their pending office visit. Embodiments allow the patient additional time to better formulate a question or questions related to the visit before seeing the doctor. Embodiments enable the doctor or clinician to move the appointment along faster and more efficiently knowing that the doctor or clinician has already explained the basics about the topic of interest via the VPP. Some of the redundancy can be reduced or eliminated and the conversation can move faster and move forward due to this process in many cases. The patient is having a value added to the process and has every opportunity to speak with their MD or clinician at their scheduled office appointment and address any concerns or questions they have. Embodiments can preserve important and beneficial aspects of the doctor-patient interaction while providing more information through a more efficient process. The VPP can be connected from any smart phone, iPad or computer. It is the VPP that allows access (e.g., through a 4-digit code, QR code, token, or other unique identifier) to be given out to the patient from the doctor's office. Embodiments provide an identifier (e.g., a 4-digit code, QR code, token, or other unique identifier) that allows any person with an electronic device with internet access to gain access to the doctor or clinician's healthcare videos.

Another important benefit of certain embodiments of the CVES is the way the program can help nurses and the workers answering phones at a healthcare office. For example, patients that have health-related issues or concerns can call the doctor's office looking for answers. In certain cases, the question can be asked to the nurse or staff answering the phones. A follow up call or putting a patient on hold is frequently considered normal, until the MD or clinician can be asked for an answer or until they can make a personal call back to the patient. By utilizing embodiments of CVES, the office records short videos (e.g., one or more videos on each of the top five to ten topics and reasons patients call the office.) At each specialty of MDs, offices routinely see the same topics and subjects brought up at the office appointments. These are advantageous topics for the MD-made videos that will have the greatest impact on their patients. Utilizing embodiments of the subject invention, the MD, clinician, or nurse records a video answering each concern and providing suggested next steps. This information can help some or all patients in between scheduling an appointment with the healthcare provider and the actual appointment. There are examples of how information can be shared in non-emergency situations from a patient having missed taking birth control pills and asking what they should do next, to a patient that has just tested positive for Covid but feels ok. In either scenario, a patient can watch a video made by their own clinician, which would give their patient information before the scheduled appointment. If in a high-volume office, 15 people in a week call with Covid, the flu or RSV, that results in 60 patients in 4 weeks or a month. If a nurse has to call back each patient and explain what to do and spends 10 minutes with each patient that would be (60 people×10 minutes=600 minutes or 10 hours. With an embodiment of the subject invention in place, this 1 nurse could have made 1 ten-minute video and redirected the patient to the VPP to enter a 4-digit code and watch a video explanation on what is the next step after testing positive for Covid, the flu or RSV. Again, the patient can watch the video multiple times and the nurse can be just as effective in helping those 60 people with the video that is recorded. The nurse can now get back to the patients in the office that the nurse missed helping for 10 hours prior to the implementation of an embodiment of the subject invention. The value recovered to replace a nurse, physician, or other clinician for 10 hours when that provider could have been in an exam room can save the office $400 if the nurse is paid $40 an hour for 10 hours. Hourly rates can be much higher based on locations, qualifications, skills, market demands, and availability. Some or all of this communication can now be directed to the VPP, enter the identifying code and watch the MD video that the office directs you to. In some embodiments, 50 patients can be on the portal at the same time, some or all getting help, watching videos from the same MD or clinician. In addition, this same system can be used at a pharmacy where a patient may have questions for the pharmacist. The same actions may be taken redirecting the patient to a VPP allowing for quicker answers leading to increased efficiency at a pharmacy 24 hours a day.

Referring now to FIG. 1C, if 8 people call into the office the first hour of a day, it can take some time to resolve their issues or get them advice. However, if they go to the VPP according to an embodiment, they can see their topic of interest addressed by a video from their MD or clinician giving advice or information that can then be further discussed at the upcoming appointment at the doctor's office. In some embodiments the patient using the VPP (1) gets the answer they were looking for, (2) gets the answer with no delay through the VPP, (3) can watch again for no fee, and (4) benefits from the YPP being available 24 hours a day to some or all patients even when the healthcare office is only open during specific days and hours.

In certain embodiments the VPP is made up of several components to which the patient can have access. First is the doctor profile, this is where a patient can receive information about their doctor and their office. Some examples would be the phone and fax number to the office. In addition, what health insurance is accepted along with what are the office hours and with which hospitals are the doctor(s) associated. Also, what languages are spoken at the office along with where the doctor got their education or degree. In addition to the library of custom-made videos, there can be a forms tab that allows patients access to certain forms that would be distributed or be asked to be filled out at the office. This gives easier access and allows for a quicker check-in at the office and adds to the efficiency and convenience needed for the modern healthcare office. There can also be a Links tab for articles or attachments to healthcare supportive information which can be displayed at the direction of the MD or clinician's office. Embodiments provide a Health Insurance Portability and Accountability Act of 1996 (HIPAA) compliant website which can allow for a more streamlined log in process for the patient, allow certain embodiments to gather and/or use patient information for provider tracking purposes on patient utilization, video utilization, and documentation purposes for possible billing codes and legal issues for the provider.

In certain embodiments, utilization of the VPP in the CVES allows for quick healthcare information leading to quick feedback in an exam room. The patient can be directed to watch a video after scheduling an appointment, at home. If the video isn't watched by the patient at home then it can be watched in an exam room minutes before the MD or clinician enters the room for the consultation and/or exam. Based on the initial nurse assessment at the office, the recommended video to watch can be set up on the patient's smartphone, or office iPad if available. The patient can be directed to watch a short video that can provide basic information and background information allowing for the real in person appointment with the MD or clinician to progress further than otherwise possible. The patient can be better prepared as they have had some degree of front-loading healthcare information on the topic of interest. As the doctor enters the exam room and begins the interaction, embodiments provide that the patient can review the information just viewed from their clinician. This allows for immediate feedback, positive or negative, and allows for engagement between the patient and doctor. Most people believe in their MD so when the doctor or clinician gives advice it is generally taken without big push back. This can help facilitate the next appropriate step if needed in each individual case. In addition, embodiments provide a HIPAA compliant website that allows for a more streamlined log in process for the patient, gathering and/or use of patient information for provider tracking purposes on patient utilization, video utilization, and documentation purposes for possible billing codes and legal issues for the provider in real time.

There is presently a shortage of healthcare workers in the US. New options to increase efficiency and to help healthcare offices counsel and educate patients provide an opportunity. After watching a video on the topic or the reason for their visit, the patient can, in real time, have a better understanding of the basics and be able to better articulate a thought-out question or questions by simply having a few extra minutes (or in certain embodiments, up to a week or more) before seeing the doctor or clinician in person. This healthcare video made by their MD or clinician can engage the patient and help them to understand if this information correlates with the symptoms or reasons for their healthcare appointment. If the patient does not believe the information correlates to their symptoms or situation, then this video driven engagement allows for communication in to occur more readily in a new direction. Either way, embodiments of the VPP and CVES are effective and can help save time. When the patient watches the video at the direction of their MD or clinician, everyone is working and moving in the same direction and more can be accomplished.

Another exemplary embodiment would be in ophthalmology when patients need to choose an Intraocular Lens (IOL) for cataract surgery. Currently there are multiple options a patient can choose from. These options vary on insurance coverage, out of pocket cost, patients' choice of visual outcome, etc. Providers and staff spend a lot of time with each patient discussing the options. This time spent can be saved by utilizing an embodiment of the VPP wherein the provider creates videos on cataract surgery, possible lens options, outcomes desired, and cost. The patient can enter the providers VPP and view a series of provider made videos discussing the options. The patient is now more informed about their upcoming surgery, IOL options, potential outcomes, and cost. By utilizing the CVES the provider, staff and patient can now have a focused discussion based on the information the patient received from their provider utilizing the VPP. This will save time for both the provider and staff, while creating a more educational environment for the patient.

In some embodiments the face-to-face interaction between the provider and patient happens at the clinic level. The provider utilizes the CVES and has the patient watch the videos created by the provider through the VPP prior to the patient/provider face to face interaction on the topic assigned by the provider or staff. The VPP improves communication by educating the patient on the topic prior to the face-to-face interaction with the provider. The patient education creates a more focused discussion on how the provider treats a certain situation and allows the patient to ask more focused questions. Embodiments save time for the provider and staff in clinic by having a patient educated in what and how their provider will handle the reason for the visit. Embodiments inhibit or limit call-backs because the patient can review the topic again on the providers VPP and can eliminate some of the patients preconceived online searches of websites and other random videos. All doctors do not treat all situations the same, this is why the providers VPP can help their patients understand how and why the provider is choosing to provide care and information in a certain way based on the providers' video. Some people are shy and talking about a subject or topic can be embarrassing or awkward. In certain situations, according to an embodiment, watching a video on sexually transmitted diseases (STDs) or sexually transmitted infections (STIs) privately at home or in an exam room can be informative and allow the patient to consider having the next step in a conversation about this or any subject or topic.

In certain embodiments, the video creation process allows the healthcare provider to have advantageous and beneficial options including location, timing, and method of video creation. The healthcare provider can create and record multiple videos in the privacy of their office by recording with a smart phone, iPad, computer camera, or any video recording camera in various formats, including HD. The videos can then be uploaded to the VPP. This can be accomplished by following simple instructions provided on the VPP once the healthcare provider signs up for the service. Another option in certain embodiments is to have the healthcare provider hire a videographer and record their short videos. This is a service that can be provided by the provider of the CVES and VPP, by the healthcare provider themselves, or by a third party. The videographer can take care of some or all administrative steps and tasks of transferring and uploading along with marking and titling the videos. Numerous improvements and advantages can come from services provided according to embodiments of the subject invention, including faster and more efficient creation, cataloging, and organization of videos.

Embodiments allow a multiplicity of patients to receive the same healthcare information from their doctor, clinician, and/or healthcare provider so that their baseline understanding is delivered to a known standard.

Embodiments allow one or more patients to more readily gain additional access to healthcare information from their doctor, clinician, and/or healthcare provider.

Embodiments allow the patient to get information that they're interested in, faster and at their convenience from their doctor, clinician, and/or healthcare provider.

Embodiments allow the patient to better prepare for their office visit.

Embodiments allow for the doctor, clinician, and/or healthcare provider the ability to reduce some redundant information that is discussed in their office (e.g., topics the healthcare provider can discuss with one or more patients as frequently as every month, every week, or even every day.)

By reducing the time spent to deliver this redundant information, the doctor, clinician, and/or healthcare provider can save time (e.g., between 1 minute and 30 minutes, including increments, combinations, and ranges thereof) with each of their patients, while increasing the quality and reliability of the information delivery to each individual patient.

In certain embodiments, when the doctor, clinician, and/or healthcare provider can save a few minutes, multiplied by a number of their patients (e.g., between 1 patient and 200 patients, including increments, combinations, and ranges thereof) seen in a week, this time can add up to significant hours saved per week.

Embodiments provide systems and methods wherein when patients call in to the office by phone, they can be re-directed to the VPP. The answers to commonly asked questions can be accessible 24 hours a day as compared to just when the office is open and answering the phone.

In addition, with the skilled labor (e.g., nurse, therapist, nursing assistant, etc.) shortages experienced in the United States and other countries of the world, the video portal can be used to reliably counsel and educate patients on different healthcare related topics saving valuable time on and off the phone.

According to certain embodiments, the video portal can be used as a substitute to nurses that provide counseling and education separate from the doctor or clinician.

Taking advantage of the numerous benefits and advantages of embodiments of the subject invention, healthcare visits can occur with significant time savings (e.g., saving 1-6 minutes on a scheduled 15 minute visit) due to the patient having just watched the video of their clinician's video on the healthcare related topic.

Assuming for example that on average a doctor or clinician visits with 90 patients in a week and saves 3 minutes per patient, embodiments can provide savings of 270 minutes or 4 hours and 30 minutes per week. A total of 18 additional patients can now be seen each week, with an average reimbursement of, for example, $100 per patient. The clinician can generate an additional $1800 per week or $90,000 per year. Certain clinicians can have higher reimbursements.

TABLE 1
Time savings and revenue grid.

| #Of | Potential minutes saved | | Potential additional | |
|---|---|---|---|---|
| Patients | per | | patients that can be | Estimated new revenue |
| seen a | patient using “Clinician | Total minutes saved by | seen with time saved | from seeing additional |
| week | Video Efficiency System” | provider per week | per week | patients |
| | (Based on a 15-minute | (Divided 15 mins | (1 patient = 15 | ($100 avg per office |
| | office visit) | each new patient) | minutes) | visit) |
| 30 | 1-6 minutes on avg | 30-180 mins/ | 2-12 patients | $200-$1200 week |
| | | .5-3 hrs | | $10K-$60K year |
| 60 | 1-6 minutes on avg | 60-360 mins/ | 4-24 patients | $400-$2400 week |
| | | 1-6 hrs | | $20K-120K year |
| 90 | 1-6 minutes on avg | 90-540 mins/ | 6-36 patients | $600-$3600 week |
| | | 1.5-9 hrs | | $30K-$180K year |
| 120 | 1-6 minutes on avg | 120-720 mins/ | 8-48 patients | $800-$4800 week |
| | | 2-12 hrs | | $40K-$240K year |

This time saved each week, by utilizing the CVES according to an embodiment of the subject invention, increases efficiency in the office, allowing the doctor, clinician, and/or healthcare provider to see more of their patients, significantly increasing revenue from those additional patients and/or allowing additional time to focus on more challenging patients. Table 1: Time savings and revenue grid for non-limiting example scenarios, represents a non-limiting example of time saved and revenue generated from seeing additional patients with this time saved in the office.

Although one benefit of videos delivered through or as part of certain embodiments of the subject invention is to better and more reliably educate patients, additional benefits include, for example, increasing the time doctors, clinicians, and/or healthcare providers have to see additional patients and/or spend with more challenged patients.

Embodiments, for example when incorporated into the practice consistently, can reduce doctors, clinicians, and/or healthcare providers time repeating information increasing available time.

According to certain embodiments, when doctors, clinicians, and healthcare providers record videos for their patients, the patients watching will have greater interest because they are recorded by their doctor, clinician or healthcare provider. Embodiments provide a greater bond and an increased credibility as compared to another video found on the internet from various sources.

Embodiments provide patient verification, documentation, and tracking of viewed doctor provided videos. In certain embodiments the VPP, utilizing the CVES, works by having the patient enter a doctor's passcode provided from the office. The doctor's passcode allows the patient to enter the doctor, clinician, and/or healthcare providers private and secure VPP.

In certain embodiments the CVES and/or VPP allows for different ways (e.g., either tracking automatically, as a configurable option for each patient, or as a selectable option for an individual patient interaction, treatment event, or specific video) to track (e.g., to support compliance, follow-up, feedback, quality improvement, individualization, personalization, risk mitigation, or litigation prevention measures) or to implicitly or explicitly choose not to track (e.g., to address patient privacy concerns, embarrassment over sensitive health topics, or reluctance on the part of the patient) which patients have logged on to the system and to document which videos or portions of videos were watched, at which dates and times.

In certain embodiments this tracking can be provided as an automatic, mandatory, or completely optional element, and can be chosen by the doctor, clinician, and/or healthcare provider, and can be used to document and confirm that a patient has or has not entered the VPP, watched one or more specific videos, or been informed of the specific information contained in any one or more videos. Embodiments provide enhanced workflows to improve follow up communication, further education, and confirmation with patients on a personalized and treatment specific basis.

As an example, if a patient claims that in the simplest of term they were not informed that salt (sodium) is bad for hypertension or that increased sugar consumption and lack of exercise can lead to diabetes, the VPP and the CVES according to an embodiment of the subject invention can provide tracking and verification of the delivery of this information.

As another example, if a patient claims that they were not informed on how to properly care for a wound, or post-procedure instructions, the VPP and the CVES according to an embodiment of the subject invention can provide tracking and verification of the delivery of this information.

Embodiments provide a reliable method by which documentation can be shown in defense of the doctor, clinician, and/or healthcare provider that instructions and guidance were provided, and undue litigation can be avoided, or reputations can be defended. Auditable and verifiable documentation provided according to embodiments of the subject invention can also potentially lead to lower mal-practice insurance.

Embodiments provide the doctor, clinician, and/or healthcare provider with a multiplicity of options (e.g., default, configurable, and fully customizable profiles) when tracking or not tracking a patient with the provided VPP and CVES.

In certain embodiments the patient enters a patient passcode that can be as simple as the present date (month, day, year-001, (example 10112022-001). The final 3 numbers (e.g., 001 in this example) can represent the individual patient that the doctor's office can identify, however, this patient identifier can be blinded, siloed, controlled, randomized, or otherwise protected so that no one else can identify the patient if the data was obtained accidentally, incorrectly, or illegally. The office can give the patient passcode to the patient at the office or over the phone before the patient enters the VPP. The office can keep track of these numbers and who the patient is when the patient pass-code is given out or distributed. At any time (e.g., at the end of the month), within the VPP, the CVES provider would have the ability to generate and print a report and provide to the doctor's office a list of which patients entered and viewed videos based on the identifying patient passcodes. Certain embodiments also provide real time feedback available to the doctor, clinician, and/or healthcare provider within the patient visit or exam room environment, thus enabling the doctor, clinician, and/or healthcare provider to confirm, reinforce, build on, or enhance communication to the patient (e.g., communicating directly in person), through the VPP (e.g., by directing the patient to watch or rewatch certain videos or sections of videos before or after leaving the exam room), or by delegating (e.g., instructing a nurse, nursing assistant, therapist, or other healthcare provider to interact with the patient on a specified topic.)

In addition, embodiments of the VPP can accept a patient's name, initials, or some other personal identifier. This can allow for easier record keeping at the Medical Doctor's Office (MDO). This option can be provided as an optional decision of the MDO.

Embodiments allow the doctor to know which patient watched the video on a certain topic. This would also document the fact that the patient watched the video on a certain topic and was given information directed by their doctor, clinician, and/or healthcare provider and would allow the doctor to be able to document that the patient received specific important information on a on a specific date. In addition, embodiments help the doctor, clinician, and/or healthcare provider to reduce or eliminate liability in litigation.

An additional tab in embodiments of the VPP allows the patient to print forms necessary from the MDO (e.g., Prior Approval form, insurance verification form, and the like).

Embodiments provide the CVES utilizing the VPP as an advanced efficiency system or process that when used with a large number of videos and video topics will engage the patient to play an active role in their healthcare and potentially save the doctor, clinician, and/or healthcare provider time in the patient office visit.

Embodiments allow the patient to be more engaged by watching their own doctor, clinician, and/or healthcare provider, such that the message given in the video will carry more weight with the patient.

Embodiments provide a CVES that is unique in structure, process, scale, and utilization. The doctor, clinician, and/or healthcare provider can save potentially a few minutes with each patient at their office visit due to the provided early viewing of a video on the topic or reason for the patient's scheduled office visit.

Embodiments further allow nurses, doctors, clinicians, healthcare providers, and office staff to save valuable time when patients call the office or clinic with redundant questions that require answers that take time and that can be accomplished by re-directing the patient to the video patient education portal where there will be a library of videos on topics of interest. The provided videos will be able to answer the patients' general questions and will save significant time for the entire healthcare provider team. When a few minutes are saved with each patient visit at the office, and multiplied by all patients seen in a week, the time saved can be significant hours per week. This time saved can be translated into seeing additional patients, generating additional new revenue.

Embodiments provide a system and method wherein some or all videos are recorded by the doctor, clinician, and/or healthcare provider, or a videographer utilizing a cell phone, computer camera, or video recording device. Instructions can be provided via the CVES or VPP on how to record and upload videos. This allows for convenience, flexibility and time management at the doctor's office or clinic.

According to certain embodiments, in an emergency (e.g., in the case of an evolving public health crisis, or in response to new research or treatments applicable to a number of patients), a physician can record a video, upload it to the video patient education portal and be able to communicate to some or all of their patients. The provided VPP allows for rapid, personalized, and discreet communication between the doctor, clinician, and/or healthcare provider and their patients unlike uncontrolled video sites available to the general public.

Additional services provided in certain embodiments include editing services that allow the videos that a doctor, clinician, and/or healthcare provider produce, record, and upload to also include beneficial value added elements including subtitles, allowing for reading of the messages being provided (e.g., by hearing impaired patients); artificial intelligence translation into other languages (e.g., so that all patients have access to healthcare information across language barriers); charts, diagrams, models, products, and specific examples can be placed on the screen or added to the video (e.g., to increase retention and understanding of the material and information provided by the doctor or clinician to the patient); additional borders or custom screens can be produced by the doctor or clinician by utilizing commercially available editing software.

Embodiments provide artificial intelligent software on the VPP to monitor, detect, and inhibit display of illegal, unprofessional, or inappropriate content and to flag, quarantine, remove such content upon detection and before viewing can occur.

The following products can be part of certain embodiments providing a CVES and can be included in a packaged process according to certain embodiments: (1) business cards with the specific doctor, clinician, and/or healthcare provider pass code for access to be given out for present or future use at the reception desk and exam room; (2) posters with individual QR codes for present use in the office exam room. Embodiments provide these and other tools to increase the usage and compliance.

Embodiments provide, for example, 1000 business cards with Name of the VPP, space for the Clinician's Passcode (e.g., an alphanumeric string, such as “XL27”) given out at an MDO and offered free for all patients.

Embodiments provide posters or marketing material for the exam rooms with one or more QR codes (e.g., specific codes for each clinician, for the practice, or for a larger organization such as a hospital or health system). Easy viewing is provided by embodiments with one or more QR codes built for the MDO which when scanned by the patient scans will take the patient directly to the VPP providing that clinician's videos in a manner that is highly advantageous for the exam room setting, where the provided technology and efficiency enhances ease of use in the exam room

Embodiments provide one or more clipboards for signing in at the doctor's office, clinic, healthcare facility, MDO, or other locations. Embodiments provide clipboards, for example as a reminder or tool to have a form signed at check-in stating that the patient watched a MD or clinician video, and to confirm the title-subject and date. Embodiments can provide the doctor or office documentation, that they provided specific instructions or guidance to a patient and can be used later to confirm those facts. An example would be instructions on what to do post-surgery. If, according to an embodiment, a MD makes a video on the proper steps to take post-surgery, and the patient can be documented watching it, then it takes some liability away from the MD or surgeon on post recovery. This can reduce liability in some instances and can be seen of great value. Embodiments can provide this service. The company can have the option to track some or all patient's video's that they watched and then timestamped. Embodiments provide tracking as an option that the healthcare provider or office can either choose or decline. Certain embodiments can explicitly exclude tracking of one or more items, offering alternative benefits including patient comfort or preference, lessened privacy concerns, and reduced overhead that can in certain cases offset the benefits of tracking.

The provided clipboards can be used to get the patients name, time, and date of sign-in and can be used to confirm that the patient visited the portal and followed the clinician's suggestion to watch the video associated with the reason for the patient's office visit or phone call. In certain embodiments an additional form on the clipboard that states the patient watched the clinician video associated with the reason for the office visit. This form can be used to confirm that the patient watched information provided by the doctor or clinician associated with the reason for the office visit. This statement on the provided form can be used to challenge a patient that states that were not informed certain facts (e.g., in the event that a patient later states otherwise through confusion, lack of recollection, or malice.)

An example of how an embodiment can change a medical outcome is a patient with diabetes that is non-compliant and has transportation issues that would lead to challenges of getting to a doctor's office. The patient after getting instructions at a medical office or clinic can rely on the VPP to watch and review videos on diabetes, keeping to a schedule, how to administer insulin correctly, when to call the office or seek emergency help, meal planning, and exercise suggestions. The videos are recorded by the patient's own MD or clinician which increases the trust factor and credibility. The patient having access to the VPP for free, 24 hours a day, 7 days a week, gives everyone an additional support tool and gives the MD a tool to help increase positive medical outcomes. Patients can watch the same video as many times as they want until they feel comfortable with the information. Certain embodiments provide advantageously short videos (e.g., less than 10 minutes long), however, can have a high impact on mobile learning. In addition, embodiments provide a HIPAA compliant system that can allow for a more streamlined log in process for the patient, and to gather and use patient information for provider tracking purposes on patient utilization, video utilization, and documentation purposes for possible billing codes and legal issues for the provider in real time.

In certain embodiments the provider's VPP can be populated with what the provider deems important to their patients, time consuming in the office, or areas of patient education the provider believes their patients would benefit from. The input (e.g., videos) into the VPP is from the patient's provider, not some random provider or website online. The information provided will be from the patient's provider or staff on how their provider handles a medical situation or care, not how some random provider or website online does. An example would be post-op drops or drop less post cataract regiment. Some ophthalmologists use post-op drops and others don't. The providers that use post-op drops use multiple combinations. Even within a multi-provider practice the providers can use different combinations (e.g., by making different choices with regard to dosing, preferred medicine, compounded vs brand, and over the counter products). Providers can prefer to go drop less and have many options to achieve this. The fact is each provider treats differently, and information provided by a random provider or information on a website online can be inconsistent with how another provider treats the patient. The provider and staff can spend valuable minutes with each patient unwinding the information and establishing how they treat post-op cataract surgery. When the provider utilizes the CVES and VPP by creating a post-op treatment protocol these situations can be minimized or in certain cases eliminated. Taking this example and extrapolating across 20 patients a day, the provider and staff can potentially save 1-5 minutes per patient or 20-100 minutes a day. This doesn't even include the amount of time that can be saved on call backs when patients have questions about the post-op treatment protocol. Many patient concerns can be answered prior to making a call to the office via the provider's VPP.

In certain embodiments the healthcare office can conduct their own surveys and assess what are the top reasons patients visit their office and the top reasons why they call their office. Every office and clinic can be different, and embodiments can be used differently. Videos recorded by the MD or clinician from the office can have the greatest impact on patients and make a personal connection. This impact can be through attentiveness and higher retention of healthcare information. This higher retention can be reinforced by also being able to watch the video unlimited times for free until the patient is comfortable with the information. People can absorb material at different speeds, having access to watch a video on nutrition a few times from their MD or clinician can have a potential positive affect on their life. Such suggestions from the personally made MD video can lead the patient down a path of being more proactive with their health. The VPP provides patients access to healthcare education and at home by giving the power to the patient to watch and learn from their personal MD or clinician. This can give some or all patients greater access to their healthcare provider and can help in getting to the next step in the patients' healthcare journey.

Embodiments of the CVES provide a comprehensive program that when followed can save the MD, clinician and office staff time in the health care office when interacting with patients without negatively impacting the quality of care. This new saved time can now be applied towards seeing additional patients each day, which can lead to greater revenue being generated. This additional time saved can also be applied towards more challenged patients. When a doctor sees 30 patients in a day, that can be 1 patient every 15 minutes for 8 hours with one half hour for lunch. If 3 minutes can be saved on average with each patient using embodiments of CVES and VPP that would be a total savings of 90 minutes a day (30 patients times 3 minutes=90 minutes saved). This 90-minutes saved allows for approximately 6 additional patients (6 patients times 15 minutes=90 minutes) to be seen during the workday. For primary care, if for example the average reimbursement is $100 per patient, this can generate an increase of $600 per day and increase patients seen by approximately 20% per day within the same time window. By implementing an embodiment of CVES and VPP the benefits can be auditable by the outcomes and additional revenue from additional patients seen each day. The level of success can be assessed on adaptation to guidelines that keep the doctor or clinician on schedule and within the average time frames. If the doctor is aiming for 12 minutes with each patient, embodiments can provide a physical, virtual, or simulated knock on the door or a silent alarm at 10 minutes so that the doctor or clinician can assess where they are in the visit and try to keep to the schedule without having a negative effect on the patient's outcome. By way of example but not limitation, these cases present averages over time so there can be more challenging appointments that occur and must be accounted for. One of the many benefits of embodiments of the subject invention is that the overall time saving does allow for focus on more challenged patients.

Embodiments provide a CVES that is unique in structure, process, scale, and utilization. The doctor, clinician, and/or healthcare provider can save a few minutes with each patient at their office visit due to the provided early viewing of a video on the topic or reason for the patient's scheduled office visit.

Embodiments further allow nurses, doctors, clinicians, healthcare providers, and office staff to save valuable time when patients call the office or clinic with redundant questions that require answers that take time and that can be accomplished by re-directing the patient to the video patient education portal where there will be a library of videos on topics of interest.

Embodiments can direct patients to the VPP, either directly or via the provider or provider's staff. The CVES helps manage providers time by utilizing the VPP and providing the access to the providers VPP to their patients.

Embodiments of the subject invention address the technical problem of providing detailed and helpful medical information to patients being challenging and requiring substantial amount of a healthcare provider's time. This problem is addressed by providing a CVES, in which an educational VPP applying a combination of advanced techniques is utilized to reliably and efficiently deliver information to one or more patients with confirmation, audit, and follow-up capabilities and the ability for the patient to be directed to an exam room where they can view the video immediately before the doctor, clinician, and/or healthcare provider enters the room.

In certain embodiments, when the MD or clinician begin to use the program, better results can be realized by staff implementing some or all tools and following the system. The process for best results can be discussed with the MDs, nurse staff, and workers answering phones as part of a training or onboarding process within the scope of certain embodiments. Beneficial processes of embodiments can include directing some or all patients at the scheduling stage, or some or all patients at the waiting in the exam room stage, to view a short video on the general topic or reason for their visit one or more times. For example, a healthcare video recorded by the MD or clinician gives the MD or clinician the opportunity to set the stage for the appointment and provide consistent background information that will help facilitate and support the visit. In a case where the patient is at home, they can go to the Portal address and enter an identification code and have access to their healthcare provider videos. In a case where the patient is at the doctor's office and in an exam room, embodiments can provide an Exam room poster with a QR Code attached. By scanning this code with the camera phone, the VPP can open. The identification code can be embedded into the Portal so the patient can, for example, click Submit and access their MDs videos in seconds from their smart phone.

Embodiments provide the doctor, clinician, and/or healthcare provider's private secure VPP, where a library of videos created for patients by and of their doctor, clinician, and/or healthcare provider are stored, cataloged, and arranged. The patient can watch the video topic assigned with their upcoming scheduled visit before the doctor, clinician, and/or healthcare provider enters the exam room.

When the MD or clinician sign up to be part of the VPP and CVES program embodiments can provide different levels of benefits based on level of participation. If the office is high-volume, they can benefit from implementing the program with a significant level of improvement in time spent and time saved. The MD or clinician can speak with the workers answering the phones. The workers can be directed to ask the patient the topic of their visit after scheduling an appointment. Based on the answer from the patient, the patient can be directed to a VPP to watch a video on the topic or reason for the doctor appointment, based on availability of the topic and subject matter. If the patient does not watch the healthcare video at home then the patient can be asked to watch a video from the patient's smart phone, before the actual appointment with the MD or clinician. This can take place when the nurse brings the patient to an exam room, while recording basic information such as blood pressure and weight. There is often a wait in the exam room so watching a 5 to 10-minute video here can make efficient use of available time for both the patient and the office. Embodiments provide 2 steps where the video watching can be requested or required: at the appointment setting stage and at the exam room stage. In certain embodiments, the patient can have an appointment after watching a video, providing an opportunity to ask the MD or clinician any questions from the video. Embodiments can provide an ability for the patient to ask or record questions on the CVES or VPP, providing advantages of improved patient interaction and transfer of information. Other embodiments can exclude any ability for the patient to ask or record questions on the CVES or VPP, providing advantages of simplified process flow and encouragement for the patient to ask questions directly of the healthcare provider at a medical appointment.

Embodiments of the subject invention provide a system and method for delivering healthcare services from an HCP to a plurality of patients in a healthcare setting. AN HCP can include a physician, physician assistant, nurse, therapist, technician, or other provider of healthcare. A healthcare setting can include a hospital system, a hospital, and ambulatory surgical center, an outpatient facility, a doctor's office or group practice office, or other facility, building, clinic, portal, website, or logical, digital, or physical location where healthcare services are delivered to patients.

Methods can comprise multiple steps and systems can comprise multiple elements, described herein by way of non-limiting examples. Creating a VPP can comprise accessing or presenting a structured user interface directing an HCP to create one or more videos connected to one or more healthcare topics or administrative topics in a database. A plurality of healthcare services to be provided to patients in the healthcare setting can comprise well-visits, advice, guidance, diagnosis, prescription, physical examination, evaluation, palpation, physical therapy, and other treatments known in the art to be provided to a patient by an HCP. A plurality of administrative procedures to be followed by patients in the healthcare setting can comprise appointment scheduling and attendance, required patient or insurance information, HIPAA compliance, office hours, contact procedures, emergency procedures, or other non-medical information that is beneficial to the operation and efficiency of a healthcare setting. For each healthcare service and for each administrative procedure, respectively, one or more video messages can be connected to the respective healthcare service or administrative procedure. By connected is meant either logical or topical connection (e.g., common subject matter), labeling or marking connection (e.g., common titles or names), or taxonomy or indexing connection (e.g., entries in a database or list).

In certain embodiments creating a service schedule for the HCP to provide healthcare services in the healthcare setting on a specified service date comprising interaction with a medical office or healthcare setting management and scheduling system or application, as known in the art. Embodiments can also provide novel functionality wherein the creation of a service schedule can be informed, changed, or structured based on VPP information (e.g., more time or less time can be scheduled for each respective appointment based on information from the VPP including but not limited to most recent viewing date by a patient of one or more healthcare of administrative videos, viewed or not-viewed status regarding a patient and one or more videos, sequence of videos viewed by a patient, questions raised by a patient, or correlation to past results of different patients after viewing a specified video. For example, if historical data shows that patients having watched all three of a first video, a second video, and a third video within the week prior to receiving a specified healthcare service were able to complete their office visit in less time as compared to patients who had not watched all three of the first video, the second video, and the third video within the week prior to receiving the specified healthcare service; then the scheduled time for a future patient to receive that specified healthcare service can be adjusted up or down accordingly.

A communication to a patient can include a name of the HCP (e.g., a last name of a physician, physician assistant, nurse, or therapist), the specified service date, the specified service time, at least one specified healthcare service, and at least one specified administrative procedure. In certain embodiments the communication also includes a first link to request, from the VPP, a first video message connected to the first healthcare service, a second link to request, from the VPP, a second video message connected to the first administrative procedure, and instructions to review, from the VPP, prior to the first specified service time on the specified service date, the first video message connected to the first healthcare service and the second video message connected to the first administrative procedure. Links can be specific to a patient, a procedure, an appointment, and a date and time. Links can include a web address, a QR code, a patient login, and other information. Links can be common among multiple patients, with different access, permissions, messaging, or actions taken depending on the specific patient login information, account, or credentials used.

Embodiments of the CVES and VPP can be configured and adapted to deliver a video message from a specific HCP before, during, or after that HCP visits with a specific patient or delivers a healthcare service to that patient. For example, the CVES and VPP can be configured and adapted such that while the HCP is scheduled to be physically in a first room with a first patient delivering a first healthcare service, a video representation of the HCP is presented to a second patient in a second room where the HCP is schedule to arrive within a certain amount of time after leaving the first patient. In certain embodiments, the second patient is provided relevant information to improve the quality and efficiency of interaction with the HCP just before meeting with the HCP and while the HCP is either engaged with first patient or in transit between the first patient and the second patient.

Embodiments provide instructions to review one or more video messages including an invitation to view the video messages (i) outside the healthcare setting prior to the specified service date, (ii) inside the clinic on the specified service date, and (iii) outside the healthcare setting after the specified service date. Instructions and invitations can include push notifications, schedule text or email messages, and passive links accessible to the patient at specified times, including 24 hours per day and 365 days per year access.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

### Example 1

In an OBGYN office, a patient comes in for the first time to get birth control. Instead of the doctor taking 15 or 20 minutes to explain all of the different options, embodiments will allow a patient to watch their doctor or clinician on video explain the different options available. This will save the doctor or clinician valuable time and make it convenient for the patient to watch the video potentially multiple times allowing them to digest the information and to focus on the top options that they are interested in and again, because of the CVES utilizing the VPP, the doctor will be able to save time by avoiding redundant conversations.

### Example 2

In a preventive health care scenario, a patient is interested in nutrition and potentially losing weight or eating healthier. Instead of receiving uncontrolled, conflicting, or out of date information from other sources, the patient will watch a video recorded by their doctor, clinician, and/or healthcare provider on the recommended and preferred steps to take to achieve healthier diet and exercise. This is valuable information that normally a doctor does not have sufficient time to spend covering with a patient in the office. In this embodiment the CVES utilizing the VPP allows the doctor or clinician to have the impact and effects of being in multiple places at once, since many patients can be watching their doctor, clinician, and/or healthcare provider on videos all at once while the doctor, clinician, and/or healthcare provider is meeting with one patient in real time. In this example the embodiment creates added value for the health care provider and multiple patients at the same time.

### Example 3

In a non-emergency situation, the patient can watch a recorded video by their doctor, clinician, and/or healthcare provider on the preferred and recommended steps to take to reduce hypertension, avoid diabetes, or avoid other specific health care challenges. By allowing the doctor, clinician, and/or healthcare provider to place, catalog, and index videos into the VPP, this embodiment allows the doctor, clinician, and/or healthcare provider to have the impact and effects of being in multiple places at once. Embodiments of the process and system for the CVES changes the approach to healthcare by incorporating efficiencies not previously available including specificity, documented consistency, confirmed delivery, personalized alignment, controlled repetition, and auditable delivery of information delivery, presentation, and messaging.

### Example 4

As patients test positive for Covid or the flu, instead of a nurse or HCP getting on the phone with every patient for 10 minutes and explaining their options and instructions, embodiments of the CVES allow their doctor, clinician, or healthcare provider to make a video giving specific, detailed, and personalized instructions and guidance on what to do next. This will allow the receptionist or nurse answering the phone to re-direct the patient to the VPP where they can watch a video recorded by their doctor, clinician, or healthcare provider. The video will give the patients guidance, support, and direction as to what the patients next step should be. The video will allow for the patient to get the exact information they would receive from their doctor, clinician, or healthcare provider, because it is recorded by them. The patient will receive a higher quality of communication since the provider can spend more time recording a video or portions of a video for all patients than they can afford to spend covering the same information for each individual patient. In this example, if a patient needs additional healthcare support, they will call back to the office and follow the steps set up by the doctor, clinician, or healthcare provider. Following this example for even a minor public health event, if 25 patients called a busy, high volume primary care office during 1 week, the CVES will save the nurse 250 minutes (25 patients times 10 minutes) on the phone. This is 4 hours and 10 minutes that the nurse can work on additional tasks or focus on more challenged patients. This will all be accomplished while also addressing, helping, and providing customized information to all 25 patients that called inquiring about what to do with a positive Covid or flu test. This embodiment of the CVES will improve efficiency in the office operations.

### Example 5

In an emerging treatment scenario, a doctor, clinician, or healthcare provider wants to promote a service or product that will provide better care for their patients and generate more revenue for their practice. The provider will record a video of their presentation in an embodiment of the CVES and invite some or all patients to watch it through an embodiment of the VPP. Each patient can choose to watch at home, in their car waiting for an appointment, or in an exam room before they see the doctor, clinician, or healthcare provider. This system again allows the doctor, clinician, or healthcare provider to have the impact and effects of being in more than one place at a time. This embodiment of the system increases the efficiency of the office, better serves patients, and helps generate new revenue from the time saved.

### Example 6

In a regularly scheduled care scenario, a patient calls to make an appointment. After the appointment is scheduled, the receptionist or nurse will direct the patient to visit the doctor, clinician, and/or healthcare provider's private and secure VPP to watch videos assigned by the patient's provider.

If the patient did not watch the video at home the patient will be directed to an exam room where they can view the video before the doctor, clinician, and/or healthcare provider enters the room. In the doctor, clinician, and/or healthcare provider's private secure VPP, there will be a library of videos created for patients by and of their doctor, clinician, and/or healthcare provider. The patient will watch the video topic that was assigned with their upcoming scheduled visit before the doctor, clinician, and/or healthcare provider enters the exam room. By the patient watching the video, absorbing basic information, and potentially formulating some questions, this will make the office visit more effective, more efficient, and in addition, the patient can be more direct, saving the doctor, clinician, and/or healthcare provider time while providing improved care to the patient in the visit.

### Example 7

A managing physician, in an effort to illustrate certain challenges faced in managing a clinic, hospital, department, or health care practice, asked four different physical therapy exercise technicians, each respectively working with and supporting patients from one of four different doctors from the same healthcare organization, to explain post-surgical recovery procedures as they would be explained to a patient. Each respective technician took the time to send a message to the respective physician, after which each respective physician took time to generate a reply, ultimately resulting in several minutes of collective time spent, while generating four different answers. None of the four respective answers was identical to any of the other respective answers. In each case, the technician was required to read a text message from the physician, interpret the message, and relay a verbal representation of the message, including visual cues or demonstrations added to communicate certain points. The entire process took about 5 minutes, with 4 exercise technicians and 4 physicians engaged, resulting in a potential time cost of about 40 person-minutes, plus the time of the managing physician waiting for the respective answers.

With an embodiment of the CVES, a multiplicity of (e.g., the 4 in this example, or 10, or 100) exercise technicians will be able to offer to a multiplicity of patients (or to a multiplicity of managing physicians) an improved video answer directly from their personal physician by providing a link to the VPP such as a business card, QR code, or web address as part of their normal process with the patient, effectively saving about 5 minutes per patient interaction.

### Example 8

A patient was preparing for hernia surgery the following day, and had been told that he would receive paperwork on the way out from the surgery. The patient anticipates his wife will read the paperwork after she drives him home from the outpatient surgical center, because he anticipates being under the effects of anesthesia following the surgery. The patient experiences stress and anxiety because he fears his wife may not understand certain aspects of the care instructions. The patient experiences further anxiety when realizing the doctor's office will be closed when he comes out of anesthesia that evening and anticipates that he will have questions to ask.

An embodiment of the subject invention will improve information flow to the patient and reduce possibility of post-surgical complications, while allowing the patient to reduce stress and anxiety by (i) having access to the information at his discretion before (e.g., at home, or on the ride to the health care setting for the procedure), during (e.g., in the waiting room, exam room, or surgical prep room), and after (e.g., back at home again that evening) the surgery; (ii) receiving information directly from his personal physician in the form of a secure video directed to a topic of interest; and (iii) having the opportunity to ask questions and give feedback on the video information provided by the VPP at any time before, during, or after the procedure.

### Example 9

A primary care physician gives a 4 digit code to a patient, linked to an embodiment of the CVES and providing access through a VPP to certain healthcare or administrative videos from the physician. The patient logs in to the VPP with a HIPAA-compliant login by patient name to watch the videos. The VPP and CVES then track videos watched, specific times, number of views, and other useful information. The physician, nurse, office staff, or other personnel then enter certain specific billing codes to a payor for patient education, as appropriate to the information provided to the patient. The CVES and VPP provide a clear record of the patient education services, allowing the physician to properly bill for services provided on a patient by patient basis and to thus avoid concerns of over or under billing.

### Example 10

A first HCP generates publicly available and searchable video content (e.g., a YouTube channel) in an effort to better serve and better communicate with their patients. In addition to being viewed by patients of the first HCP, as intended, the video content is viewed by other individuals who are not patients of the HCP. A first individual takes certain information in one or more videos out of context, applies the information their own situation without ever consulting the first HCP, and ends up with an adverse healthcare event. The first individual then files a lawsuit against the first HCP seeking monetary damages. The first HCP now has to defend the lawsuit.

A second HCP generates secure, privately available and indexed video content in an embodiment of CVES with a VPP in an effort to better serve and better communicate with their patients. The videos in the VPP are only viewable by patients of the second HCP, as intended. Further, videos are sequenced, and access controlled such that certain prerequisite videos (e.g., general hygiene and infection prevention) are required to be watched before granting access to other videos (e.g., post-surgical wound care), and detailed records of all video activity for each patient are kept by the CVES and the VPP. The video content is not viewed by other individuals who are not patients of the HCP. No lawsuit is filed.

### Example 11

A first patient, in a first home setting at a first time, watches (or is provided with an invitation to watch) a first video created by and featuring a first HCP explaining a medical topic or procedure. A second patient, in a second home setting at a second time, watches (or is provided with an invitation to watch) the first video.

The first patient, in a first room within a healthcare setting at a third time following the first time and the second time, watches (or is provided with an invitation to watch) the first video again while waiting to see the first HCP. The first HCP, at a fourth time following the third time, enters the first room, confirms the first patient has watched the first video, and provides one or more healthcare services to the first patient.

Also at the fourth time, concurrent with the first HCP providing one or more healthcare services to the first patient, a second patient, in a second room within the healthcare setting, watches (or is provided with an invitation to watch) the first video again while waiting to see the first HCP. During the fourth time, the first HCP is effectively providing value to two patients at once, as the first patient in the first room receives healthcare services directly from the first HCP, while at the same time the second patient in the second room receives information from the first HCP in video form.

At a fifth time following the fourth time, the first HCP enters the second room, confirms the second patient has watched the first video, and provides one or more healthcare services to the second patient.

In each example above, the doctor, clinician, and/or healthcare provider has saved valuable minutes and been able to visit with other patients while the patient was in an exam room reviewing the doctor, clinician, and/or healthcare provider videos through an embodiment of the subject invention. Now the in person, patient-doctor, clinician, and/or healthcare provider visit can be more effective and significantly shorter in time. The patient can review their doctor, clinician, and/or healthcare provider's videos anytime or whenever there is a question, or a patient needs a reminder of what the doctor, clinician, and/or healthcare provider discussed. This experience will occur hundreds of times or thousands of times a year in a busy office or clinic. This new time that the doctor, clinician, and/or healthcare provider and staff has saved can be focused on additional patients, generating new income or the doctor, clinician, and/or healthcare provider can focus on more challenged patients. Either way the new efficiency of the program opens opportunities to enhance patient care while improving the effective impact of their time with their doctor, clinician, and/or healthcare provider. Embodiments provide added value to the patient and increase the doctor, clinician, and/or healthcare provider earning power while improving the quality of care.

The subject invention includes, but is not limited to, the following exemplified embodiments.

Embodiment 1. A method for delivering healthcare services from a health care provider (HCP) to a plurality of patients in a healthcare setting, the method comprising the following steps:
a) creating a video patient portal (VPP), the VPP comprising:
   i. a plurality of healthcare services to be provided to patients in the healthcare setting;
   ii. a plurality of administrative procedures to be followed by patients in the healthcare setting; and
   iii. for each healthcare service and for each administrative procedure, respectively, a video message connected to the respective healthcare service or administrative procedure;
b) creating a service schedule for the HCP to provide healthcare services in the healthcare setting on a specified service date, the service schedule comprising:
   i. a first appointment with a first patient selected from the plurality of patients to receive, from the HCP in the healthcare setting, at a first specified service time, a first healthcare service selected from the plurality of healthcare services; and
   ii. a second appointment with a second patient selected from the plurality of patients to receive, from the HCP in the healthcare setting, at a second specified service time, a second healthcare service selected from the plurality of healthcare services; and
c) communicating to the first patient:
   i. a name of the HCP, the specified service date, the first specified service time, the first healthcare service, and a first administrative procedure selected from the plurality of administrative procedures;
   ii. a first link to request, from the VPP, a first video message connected to the first healthcare service;
   iii. a second link to request, from the VPP, a second video message connected to the first administrative procedure; and
   iv. instructions to review, from the VPP, prior to the first specified service time on the specified service date, the first video message connected to the first healthcare service and the second video message connected to the first administrative procedure.

Embodiment 2. The method according to Embodiment 1, further comprising:
d) communicating to the second patient:
   i. the name of the HCP, the specified service date, the second specified service time, the second healthcare service, and the first administrative procedure;
   ii. a third link to request, from the VPP, a third video message connected to the second healthcare service;
   iii. a fourth link to request, from the VPP, the second video message connected to the first administrative procedure; and
   iv. instructions to review, from the VPP prior to the second specified service time on the specified service date, the third video message connected to the second healthcare service and the second video message connected to the first administrative procedure.

Embodiment 3. The method according to any of Embodiments 1-2, further comprising:
e) delivering the first healthcare service from the HCP to the first patient; and
f) delivering the second healthcare service from the HCP to the second patient.

Embodiment 4. The method according to any of Embodiments 1-3, wherein the first video message connected to the first healthcare service comprises a visual representation of the HCP, an audio representation of the HCP, or both.

Embodiment 5. The method according to any of Embodiments 1-4, wherein the third video message connected to the second healthcare service comprises a visual representation of the HCP, an audio representation of the HCP, or both.

Embodiment 6. The method according to any of Embodiments 1-5, wherein the second video message connected to the first administrative procedure comprises a visual representation of the HCP, an audio representation of the HCP, or both.

Embodiment 7. The method according to any of Embodiments 1-6, wherein the HCP is a first HCP,
wherein the first video message connected to the first healthcare service comprises a visual representation of the first HCP, an audio representation of the first HCP, or both,
wherein the third video message connected to the second healthcare service comprises a visual representation of a second HCP, an audio representation of the second HCP, or both, and
wherein the second video message connected to the first administrative procedure comprises at least one representation selected from the group consisting of: a visual representation of the first HCP, an audio representation of the first HCP, a visual representation of the second HCP, an audio representation of the second HCP, a visual representation of a representative of the healthcare setting, and an audio representation of the representative of the healthcare setting.

Embodiment 8. The method according to any of Embodiments 1-7, wherein the communicating to the second patient the instructions to review the third video message and the second video message occurs during the delivering the first healthcare service from the HCP to the first patient.

Embodiment 9. The method according to any of Embodiments 1-8, wherein the communicating to the first patient the instructions to review the first video message and the second video message comprises an invitation to view one or both of the first video message and the second video message (i) outside the healthcare setting prior to the specified service date, (ii) inside the healthcare setting on the specified service date, and (iii) outside the healthcare setting after the specified service date.

Embodiment 10. The method according to any of Embodiments 1-9, wherein the communicating to the second patient the instructions to review the third video message and the second video message comprises an invitation to view one or both of the third video message and the second video message (i) outside the healthcare setting prior to the specified service date, (ii) inside the clinic on the specified service date, and (iii) outside the healthcare setting after the specified service date.

Embodiment 11. A system for delivering healthcare services from a health care provider (HCP) to a plurality of patients in a healthcare setting, the system comprising:
a) a video patient portal (VPP), the VPP comprising:
   i. a plurality of healthcare services to be provided to patients in the healthcare setting;
   ii. a plurality of administrative procedures to be followed by patients in the healthcare setting; and
   iii. for each healthcare service and for each administrative procedure, respectively, a video message connected to the respective healthcare service or administrative procedure;
b) a service schedule for the HCP to provide healthcare services in the healthcare setting on a specified service date, the service schedule comprising:
   i. a first appointment with a first patient selected from the plurality of patients to receive, from the HCP in the healthcare setting at a first specified service time, a first healthcare service selected from the plurality of healthcare services; and
   ii. a second appointment with a second patient selected from the plurality of patients to receive, from the HCP in the healthcare setting at a second specified service time, a second healthcare service selected from the plurality of healthcare services; and
c) a communication to the first patient, the communication to the first patient comprising:
   i. a name of the HCP, the specified service date, the first specified service time, the first healthcare service, and a first administrative procedure selected from the plurality of administrative procedures;
   ii. a first link to request, from the VPP, a first video message connected to the first healthcare service;
   iii. a second link to request, from the VPP, a second video message connected to the first administrative procedure; and
   iv. instructions to review, from the VPP prior to the first specified service time on the specified service date, the first video message connected to the first healthcare service and the second video message connected to the first administrative procedure.

Embodiment 12. The system according to Embodiment 11, further comprising:
d) a communication to the second patient, the communication to the second patient comprising:
   i. the name of the HCP, the specified service date, the second specified service time, the second healthcare service, and the first administrative procedure;
   ii. a third link to request, from the VPP, a third video message connected to the second healthcare service;
   iii. a fourth link to request, from the VPP, the second video message connected to the first administrative procedure; and
   iv. instructions to review, from the VPP prior to the second specified service time on the specified service date, the third video message connected to the second healthcare service and the second video message connected to the first administrative procedure.

Embodiment 13. The system according to any of Embodiments 11-12, further comprising:
e) a record of delivering the first healthcare service from the HCP to the first patient; and
f) a record of delivering the second healthcare service from the HCP to the second patient.

Embodiment 14. The system according to any of Embodiments 11-13, wherein the first video message connected to the first healthcare service comprises a visual representation of the HCP, an audio representation of the HCP, or both.

Embodiment 15. The system according to any of Embodiments 11-14, wherein the third video message connected to the second healthcare service comprises a visual representation of the HCP, an audio representation of the HCP, or both.

Embodiment 16. The system according to any of Embodiments 11-15, wherein the second video message connected to the first administrative procedure comprises a visual representation of the HCP, an audio representation of the HCP, or both.

Embodiment 17. The system according to any of Embodiments 11-16, wherein the HCP is a first HCP,
wherein the first video message connected to the first healthcare service comprises a visual representation of the first HCP, an audio representation of the first HCP, or both,
wherein the third video message connected to the second healthcare service comprises a visual representation of a second HCP, an audio representation of the second HCP, or both, and
wherein the second video message connected to the first administrative procedure comprises at least one representation selected from the group consisting of: a visual representation of the first HCP, an audio representation of the first HCP, a visual representation of the second HCP, an audio representation of the second HCP, a visual representation of a representative of the healthcare setting, and an audio representation of the representative of the healthcare setting.

Embodiment 18. The system according to any of Embodiments 11-17, wherein the communication to the second patient comprising the instructions to review the third video message and the second video message is scheduled for delivery during the delivering the first healthcare service from the HCP to the first patient.

Embodiment 19. The system according to any of Embodiments 11-18, wherein the communication to the first patient comprising the instructions to review the first video message and the second video message comprises an invitation to view one or both of the first video message and the second video message (i) outside the healthcare setting prior to the specified service date, (ii) inside the healthcare setting on the specified service date, and (iii) outside the healthcare setting after the specified service date.

Embodiment 20. A system for delivering healthcare services from a health care provider (HCP) to a plurality of patients in a healthcare setting, the system comprising:
a) a video patient portal (VPP), the VPP comprising:
   i. a plurality of healthcare services to be provided to patients in the healthcare setting;
   ii. a plurality of administrative procedures to be followed by patients in the healthcare setting; and
   iii. for each healthcare service and for each administrative procedure, respectively, a video message connected to the respective healthcare service or administrative procedure;
b) a service schedule for the HCP to provide healthcare services in the healthcare setting on a specified service date, the service schedule comprising:
   i. a first appointment with a first patient selected from the plurality of patients to receive, from the HCP in the healthcare setting at a first specified service time, a first healthcare service selected from the plurality of healthcare services; and
   ii. a second appointment with a second patient selected from the plurality of patients to receive, from the HCP in the healthcare setting at a second specified service time, a second healthcare service selected from the plurality of healthcare services;
c) a communication to the first patient, the communication to the first patient comprising:
   i. a name of the HCP, the specified service date, the first specified service time, the first healthcare service, and a first administrative procedure selected from the plurality of administrative procedures;
   ii. a first link to request, from the VPP, a first video message connected to the first healthcare service;
   iii. a second link to request, from the VPP, a second video message connected to the first administrative procedure; and
   iv. instructions to review, from the VPP prior to the first specified service time on the specified service date, the first video message connected to the first healthcare service and the second video message connected to the first administrative procedure;
d) a communication to the second patient, the communication to the second patient comprising:
   i. the name of the HCP, the specified service date, the second specified service time, the second healthcare service, and the first administrative procedure;
   ii. a third link to request, from the VPP, a third video message connected to the second healthcare service;
   iii. a fourth link to request, from the VPP, the second video message connected to the first administrative procedure; and
   iv. instructions to review, from the VPP prior to the second specified service time on the specified service date, the third video message connected to the second healthcare service and the second video message connected to the first administrative procedure;
e) a record of delivering the first healthcare service from the HCP to the first patient; and
f) a record of delivering the second healthcare service from the HCP to the second patient,
   wherein the HCP is a first HCP,
   wherein the first video message connected to the first healthcare service comprises a visual representation of the first HCP, an audio representation of the first HCP, or both,
   wherein the third video message connected to the second healthcare service comprises a visual representation of a second HCP, an audio representation of the second HCP, or both,
   wherein the second video message connected to the first administrative procedure comprises at least one representation selected from the group consisting of: a visual representation of the first HCP, an audio representation of the first HCP, a visual representation of the second HCP, an audio representation of the second HCP, a visual representation of a representative of the healthcare setting, and an audio representation of the representative of the healthcare setting,
   wherein the communication to the second patient comprising the instructions to review the third video message and the second video message is scheduled for delivery during the delivering the first healthcare service from the HCP to the first patient,
   wherein the communication to the first patient comprising the instructions to review the first video message and the second video message comprises an invitation to view one or both of the first video message and the second video message (i) outside the healthcare setting prior to the specified service date, (ii) inside the healthcare setting on the specified service date, and (iii) outside the healthcare setting after the specified service date, and
   wherein the communication to the second patient the instructions to review the third video message and the second video message comprises an invitation to view one or both of the third video message and the second video message (i) outside the healthcare setting; prior to the specified service date, (ii) inside the healthcare setting on the specified service date, and (iii) outside the healthcare setting after the specified service date.

It should be understood that the examples and embodiments described herein are for illustrative purposes only and that various modifications or changes in light thereof will be suggested to persons skilled in the art and are to be included within the spirit and purview of this application and the scope of the appended claims.

All patents, patent applications, provisional applications, and other publications referred to or cited herein are incorporated by reference in their entirety, including all figures and tables, to the extent they are not inconsistent with the explicit teachings of this specification.

What is claimed is:

1. A method for delivering healthcare services from a health care provider (HCP) to a plurality of patients in a healthcare setting, the method comprising the following steps:
g) creating a video patient portal (VPP), the VPP comprising:
iv. a plurality of healthcare services to be provided to patients in the healthcare setting;
v. a plurality of administrative procedures to be followed by patients in the healthcare setting; and
vi. for each healthcare service and for each administrative procedure, respectively, a video message connected to the respective healthcare service or administrative procedure;
h) creating a service schedule for the HCP to provide healthcare services in the healthcare setting on a specified service date, the service schedule comprising:
iii. a first appointment with a first patient selected from the plurality of patients to receive, from the HCP in the healthcare setting, at a first specified service time, a first healthcare service selected from the plurality of healthcare services; and
iv. a second appointment with a second patient selected from the plurality of patients to receive, from the HCP in the healthcare setting, at a second specified service time, a second healthcare service selected from the plurality of healthcare services; and
i) communicating to the first patient:
v. a name of the HCP, the specified service date, the first specified service time, the first healthcare service, and a first administrative procedure selected from the plurality of administrative procedures;
vi. a first link to request, from the VPP, a first video message connected to the first healthcare service;
vii. a second link to request, from the VPP, a second video message connected to the first administrative procedure; and
viii. instructions to review, from the VPP, prior to the first specified service time on the specified service date, the first video message connected to the first healthcare service and the second video message connected to the first administrative procedure.

2. The method according to claim 1, further comprising:
j) communicating to the second patient:
v. the name of the HCP, the specified service date, the second specified service time, the second healthcare service, and the first administrative procedure;
vi. a third link to request, from the VPP, a third video message connected to the second healthcare service;
vii. a fourth link to request, from the VPP, the second video message connected to the first administrative procedure; and
viii. instructions to review, from the VPP prior to the second specified service time on the specified service date, the third video message connected to the second healthcare service and the second video message connected to the first administrative procedure.

3. The method according to claim 2, further comprising:
k) delivering the first healthcare service from the HCP to the first patient; and
l) delivering the second healthcare service from the HCP to the second patient.

4. The method according to claim 3, wherein the first video message connected to the first healthcare service comprises a visual representation of the HCP, an audio representation of the HCP, or both.

5. The method according to claim 4, wherein the third video message connected to the second healthcare service comprises a visual representation of the HCP, an audio representation of the HCP, or both.

6. The method according to claim 5, wherein the second video message connected to the first administrative procedure comprises a visual representation of the HCP, an audio representation of the HCP, or both.

7. The method according to claim 3, wherein the HCP is a first HCP,
wherein the first video message connected to the first healthcare service comprises a visual representation of the first HCP, an audio representation of the first HCP, or both,
wherein the third video message connected to the second healthcare service comprises a visual representation of a second HCP, an audio representation of the second HCP, or both, and
wherein the second video message connected to the first administrative procedure comprises at least one representation selected from the group consisting of: a visual representation of the first HCP, an audio representation of the first HCP, a visual representation of the second HCP, an audio representation of the second HCP, a visual representation of a representative of the healthcare setting, and an audio representation of the representative of the healthcare setting.

8. The method according to claim 3, wherein the communicating to the second patient the instructions to review the third video message and the second video message occurs during the delivering the first healthcare service from the HCP to the first patient.

9. The method according to claim 2, wherein the communicating to the first patient the instructions to review the first video message and the second video message comprises an invitation to view one or both of the first video message and the second video message (i) outside the healthcare setting prior to the specified service date, (ii) inside the healthcare setting on the specified service date, and (iii) outside the healthcare setting after the specified service date.

10. The method according to claim 9, wherein the communicating to the second patient the instructions to review the third video message and the second video message comprises an invitation to view one or both of the third video message and the second video message (i) outside the healthcare setting prior to the specified service date, (ii) inside the clinic on the specified service date, and (iii) outside the healthcare setting after the specified service date.

11. A system for delivering healthcare services from a health care provider (HCP) to a plurality of patients in a healthcare setting, the system comprising:
g) a video patient portal (VPP), the VPP comprising:
iv. a plurality of healthcare services to be provided to patients in the healthcare setting;
v. a plurality of administrative procedures to be followed by patients in the healthcare setting; and
vi. for each healthcare service and for each administrative procedure, respectively, a video message connected to the respective healthcare service or administrative procedure;
h) a service schedule for the HCP to provide healthcare services in the healthcare setting on a specified service date, the service schedule comprising:
iii. a first appointment with a first patient selected from the plurality of patients to receive, from the HCP in the healthcare setting at a first specified service time, a first healthcare service selected from the plurality of healthcare services; and
iv. a second appointment with a second patient selected from the plurality of patients to receive, from the HCP in the healthcare setting at a second specified service time, a second healthcare service selected from the plurality of healthcare services; and
i) a communication to the first patient, the communication to the first patient comprising:
v. a name of the HCP, the specified service date, the first specified service time, the first healthcare service, and a first administrative procedure selected from the plurality of administrative procedures;
vi. a first link to request, from the VPP, a first video message connected to the first healthcare service;
vii. a second link to request, from the VPP, a second video message connected to the first administrative procedure; and
viii. instructions to review, from the VPP prior to the first specified service time on the specified service date, the first video message connected to the first healthcare service and the second video message connected to the first administrative procedure.

12. The system according to claim 11, further comprising:
j) a communication to the second patient, the communication to the second patient comprising:
v. the name of the HCP, the specified service date, the second specified service time, the second healthcare service, and the first administrative procedure;
vi. a third link to request, from the VPP, a third video message connected to the second healthcare service;
vii. a fourth link to request, from the VPP, the second video message connected to the first administrative procedure; and
viii. instructions to review, from the VPP prior to the second specified service time on the specified service date, the third video message connected to the second healthcare service and the second video message connected to the first administrative procedure.

13. The system according to claim 12, further comprising:
k) a record of delivering the first healthcare service from the HCP to the first patient; and
l) a record of delivering the second healthcare service from the HCP to the second patient.

14. The system according to claim 13, wherein the first video message connected to the first healthcare service comprises a visual representation of the HCP, an audio representation of the HCP, or both.

15. The system according to claim 14, wherein the third video message connected to the second healthcare service comprises a visual representation of the HCP, an audio representation of the HCP, or both.

16. The system according to claim 15, wherein the second video message connected to the first administrative procedure comprises a visual representation of the HCP, an audio representation of the HCP, or both.

17. The system according to claim 13, wherein the HCP is a first HCP,
wherein the first video message connected to the first healthcare service comprises a visual representation of the first HCP, an audio representation of the first HCP, or both,
wherein the third video message connected to the second healthcare service comprises a visual representation of a second HCP, an audio representation of the second HCP, or both, and
wherein the second video message connected to the first administrative procedure comprises at least one representation selected from the group consisting of: a visual representation of the first HCP, an audio representation of the first HCP, a visual representation of the second HCP, an audio representation of the second HCP, a visual representation of a representative of the healthcare setting, and an audio representation of the representative of the healthcare setting.

18. The system according to claim 13, wherein the communication to the second patient comprising the instructions to review the third video message and the second video message is scheduled for delivery during the delivering the first healthcare service from the HCP to the first patient.

19. The system according to claim 12, wherein the communication to the first patient comprising the instructions to review the first video message and the second video message comprises an invitation to view one or both of the first video message and the second video message (i) outside the healthcare setting prior to the specified service date, (ii) inside the healthcare setting on the specified service date, and (iii) outside the healthcare setting after the specified service date.

20. A system for delivering healthcare services from a health care provider (HCP) to a plurality of patients in a healthcare setting, the system comprising:
g) a video patient portal (VPP), the VPP comprising:
iv. a plurality of healthcare services to be provided to patients in the healthcare setting;
v. a plurality of administrative procedures to be followed by patients in the healthcare setting; and
vi. for each healthcare service and for each administrative procedure, respectively, a video message connected to the respective healthcare service or administrative procedure;
h) a service schedule for the HCP to provide healthcare services in the healthcare setting on a specified service date, the service schedule comprising:
iii. a first appointment with a first patient selected from the plurality of patients to receive, from the HCP in the healthcare setting at a first specified service time, a first healthcare service selected from the plurality of healthcare services; and
iv. a second appointment with a second patient selected from the plurality of patients to receive, from the HCP in the healthcare setting at a second specified service time, a second healthcare service selected from the plurality of healthcare services;
i) a communication to the first patient, the communication to the first patient comprising:
v. a name of the HCP, the specified service date, the first specified service time, the first healthcare service, and a first administrative procedure selected from the plurality of administrative procedures;
vi. a first link to request, from the VPP, a first video message connected to the first healthcare service;
vii. a second link to request, from the VPP, a second video message connected to the first administrative procedure; and
viii. instructions to review, from the VPP prior to the first specified service time on the specified service date, the first video message connected to the first healthcare service and the second video message connected to the first administrative procedure;
j) a communication to the second patient, the communication to the second patient comprising:
V. the name of the HCP, the specified service date, the second specified service time, the second healthcare service, and the first administrative procedure;
vi. a third link to request, from the VPP, a third video message connected to the second healthcare service;
vii. a fourth link to request, from the VPP, the second video message connected to the first administrative procedure; and
viii. instructions to review, from the VPP prior to the second specified service time on the specified service date, the third video message connected to the second healthcare service and the second video message connected to the first administrative procedure;
k) a record of delivering the first healthcare service from the HCP to the first patient; and
l) a record of delivering the second healthcare service from the HCP to the second patient,
wherein the HCP is a first HCP,
wherein the first video message connected to the first healthcare service comprises a visual representation of the first HCP, an audio representation of the first HCP, or both,
wherein the third video message connected to the second healthcare service comprises a visual representation of a second HCP, an audio representation of the second HCP, or both,
wherein the second video message connected to the first administrative procedure comprises at least one representation selected from the group consisting of: a visual representation of the first HCP, an audio representation of the first HCP, a visual representation of the second HCP, an audio representation of the second HCP, a visual representation of a representative of the healthcare setting, and an audio representation of the representative of the healthcare setting,
wherein the communication to the second patient comprising the instructions to review the third video message and the second video message is scheduled for delivery during the delivering the first healthcare service from the HCP to the first patient,
wherein the communication to the first patient comprising the instructions to review the first video message and the second video message comprises an invitation to view one or both of the first video message and the second video message (i) outside the healthcare setting prior to the specified service date, (ii) inside the healthcare setting on the specified service date, and (iii) outside the healthcare setting after the specified service date, and
wherein the communication to the second patient the instructions to review the third video message and the second video message comprises an invitation to view one or both of the third video message and the second video message (i) outside the healthcare setting; prior to the specified service date, (ii) inside the healthcare setting on the specified service date, and (iii) outside the healthcare setting after the specified service date.
